(19) United States
(12) Patent Application Publication
(10) Pub. No.: US 2018/0161389 A1
(43) Pub. Date: Jun. 14, 2018

(54) R-SPONDIN AGONIST-MEDIATED HAIR GROWTH

(71) Applicants: The Board of Trustees of the Leland Stanford Junior University, Stanford, CA (US); Ulixa, Inc., San Francisco, CA (US)

(72) Inventors: Jill Helms, Stanford, CA (US); Girija Dhamdhere, San Jose, CA (US); Andrew A. Smith, San Jose, CA (US); Arie Abo, Oakland, CA (US); Anthony E. Oro, Stanford, CA (US)

(21) Appl. No.: 15/540,865

(22) PCT Filed: Jan. 8, 2016
(86) PCT No.: PCT/US16/12735

§ 371 Date: Jun. 29, 2017

Related U.S. Application Data
(60) Provisional application No. 62/101,712, filed on Jan. 9, 2015.
(60) Provisional application No. 62/275,742, filed on Jan. 6, 2016.

(51) Int. Cl. A61K 38/08; A61K 38/17; A61K 8/64; A61K 9/00; A61P 43/00

(52) U.S. Cl. CPC A61K 38/08; A61K 38/1709; A61P 43/00; A61K 9/0019; A61K 8/64

(57) ABSTRACT

Cosmetic skin and hair care compositions for enhancing the growth and appearance of mammalian hair in an individual are provided, which compositions comprise a dose of an R-spondin agonist effective to promote anagen phase of the hair cycle.

### CROSS-REFERENCE

This application is a 371 application and claims the benefit of PCT Application No. PCT/US2016/012735, filed Jan. 8, 2016, which claims the benefit of U.S. Provisional Patent Application Ser. No. 62/101,712, filed Jan. 9, 2015, and U.S. Provisional Patent Application Ser. No. 62/275,742, filed Jan. 6, 2016, each of which is incorporated by reference herein in its entirety.

### BACKGROUND OF THE INVENTION

Adult stem cells are multipotent cells capable of executing specific differentiation programs in response to injury or key environmental signals. Many if not all adult organs possess a small number of stem cells, providing the promise of the ability to regenerate organs that are lost through injury or disease. In many tissues, multipotent stem cells are found within specific tissue niches of support cells. These niches contain specific extrinsic and intrinsic cues and act to provide regulatory signals that help pattern tissue self-renewal, proliferation or differentiation. While the relationship between stem cells and niches are known, the controls that regulate the interactions between the cells are an area of active investigation.

The hair follicle is an excellent system to study adult stem cells because of its cycle dependency and relatively short switches between growth and destruction phases. Hair follicles have two parts; one part is a permanent part that has sebaceous glands and the stem cell containing, or bulge, region. The lower or dynamic part goes through genetically controlled cycles of active growth (anagen), destruction phase (catagen) and resting phase (telogen). The timing of each of the cycle phases is exquisitely controlled and varies with environmental controls such as day length and temperature or internal controls such as metabolic or hormone status. During the resting or telogen phase, stem cells are quiescent and remain non-proliferative until the start of the next growth or anagen phase, up to three weeks later in the mouse.

The hair follicle is a complex organ composed of seven differentiation-specific tissue layers including the outer root sheath (ORS), inner root sheath (IRS) and hair shaft (HS). Hair follicle stem cells reside in the bulge region, below the sebaceous gland, in the permanent portion of the hair follicle. It has been proposed that in early anagen phase a signal from dermal papilla activates stem cells in the bulge. This causes proliferation of these cells and subsequently causes downgrowth. As the dermal papilla moves away from the bulge, the stem cells in the bulge return to their quiescent state. Stem cells in the bulge region can also give rise to epidermis in a wounding injury. Investigations over the past decade have elucidated the major signaling regulators controlling the hair cycle, where two of the central pathways for stimulating anagen are the Wnt and Shh pathways.

Hair follicle stem cells are of interest for cosmetic, as well as therapeutic, purposes. For example, androgenic alopecia is the single largest type of recognizable alopecia to affect both men and women, primarily of Caucasian origin. Androgenic alopecia or common baldness represents 99 percent of all cases of hair loss. The condition is characterized by the gradual conversion of terminal hair to short, wispy, colorless vellus hair.

In 1980, the reversal of androgenic alopecia in a male patient receiving minoxidil for hypertension was revealed and minoxidil has since been used to promote hair growth, most commonly by topical application. Minoxidil's vasodilating effect on the scalp is one of the proposed mechanisms by which minoxidil promotes hair growth. However, despite its popularity, minoxidil has not performed in a completely satisfactory fashion in promoting hair growth in all target populations. While minoxidil has been shown to stimulate some hair growth at the apex region of the scalp, hair growth at the frontal region of the scalp, for the most part, has not been shown to be improved by minoxidil treatment alone.

Each year, more than $2B is spent worldwide on surgical procedures for hair loss. The problem is not restricted to male baldness: In addition to 35M men, 20-30M women in the U.S. are also affected by hair loss. In addition, hair loss occurs as a consequence of chemotherapy, radiation, and immune suppression medications. Although hair growth usually returns when these therapies cease, some patients become permanently bald.

Current treatment options for other types of hair loss are limited; they include topical products, such as minoxidil, which retards hair shedding (effluvium) but does not stimulate new hair growth; and finasteride, which reduces testosterone conversion and also hinders effluvium. Hair transplants, where hair follicles (HF) are harvested from one part of the scalp and moved to another, are largely effective but upon harvesting of the hair follicles, a subset of them undergo apoptosis. Consequently, when those follicles are transplanted the hair falls out.

There is a continuing desire to treat hair loss. The present invention addresses this need.

### SUMMARY OF THE INVENTION

There exists a considerable need for alternative therapeutics that promote hair growth. The present invention addresses this need and provides additional advantages. In one aspect, the present invention provides a cosmetic composition comprising: an Rspo agonist in an amount effective to promote the anagen phase of hair production; and a cosmetically acceptable vehicle. In some embodiments, promoting the anagen phase comprises increasing the length of the anagen phase. In some embodiments, the promoting the anagen phase comprises increasing hair growth. In some embodiments, the promoting anagen phase delays onset of catagen phase.

In some embodiments, the Rspo agonist is a mammalian R-spondin polypeptide. In some embodiments, the mammalian R-spondin polypeptide is a human R-spondin polypeptide. In some embodiments, the R-spondin polypeptide is R-spondin2. In some embodiments, the Rspo agonist has at least about 30%, at least about 40%, at least about 50%, at least about 60%, at least about 70%, at least about 80%, at least about 90%, or at least about 95% sequence identity to at least one of the amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9. In some embodiments, the Rspo agonist is as set forth in SEQ ID NO: 1 or SEQ ID NO: 2.

In some embodiments, the Rspo agonist comprises at least about 40%, at least about 50%, at least about 60%, at least about 70%, at least about 80%, at least about 90%, or at least about 95% sequence identity to at least one of the amino acid sequences as set forth in SEQ ID NO:1 or SEQ ID NO:2. In some embodiments, the Rspo agonist comprises at least a 10 amino acid sequence that is identical to SEQ ID NO: 1 or SEQ ID NO: 2. In some embodiments, the Rspo agonist comprises at least a 20 amino acid sequence that is identical to SEQ ID NO: 1 or SEQ ID NO: 2. In some embodiments, the Rspo agonist comprises no more than a 10 amino acid sequence that is identical to SEQ ID NO: 1 or SEQ ID NO: 2. In some embodiments, the Rspo agonist comprises no more than a 20 amino acid sequence that is identical to SEQ ID NO: 1 or SEQ ID NO: 2. In some embodiments, the Rspo agonist comprises from about 40% to about 95% sequence identity to at least one of the amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9. In some embodiments, the Rspo agonist comprises from about 40% to about 95% sequence identity to at least one of the amino acid sequences as set forth in SEQ ID NO:1 or SEQ ID NO:2.

In some embodiments, the Rspo agonist comprises from about 40 to about 200 amino acids of a mammalian Rspo polypeptide. In some embodiments, the Rspo agonist comprises from about 50 to about 180 amino acids of a mammalian Rspo polypeptide.

In some embodiments the cosmetic composition further comprises one or more permeation enhancers. In some embodiments, the cosmetic composition comprises from about 0.01 to about 5.0 mg/mL of the Rspo agonist. In some embodiments, the cosmetic composition comprises from about 0.5 to about 3 mg/mL of the Rspo agonist. In some embodiments, the cosmetically acceptable vehicle is selected from diluents, dispersants, and carriers or a combination thereof.

In one aspect, the present invention provides a patch for application to the skin, wherein the patch comprises any cosmetic composition disclosed herein.

In one aspect, the present invention provides a method for treating a condition of the skin, scalp or hair of a subject in need thereof, comprising applying to the skin, scalp or hair of the subject any cosmetic composition disclosed herein. In some embodiments, the condition is alopecia. In some embodiments, the condition is androgenic alopecia. In some embodiments, the condition is caused by a vitamin deficiency, an iron deficiency, infection, chemotherapy, anabolic steroids, oral contraceptives or trauma.

In some embodiments, the subject is human. In some embodiments, the applying comprises one or more techniques selected from non-cavitational ultrasound, electroporation, cavitational ultrasound, thermal ablation, and microdermabrasion to enhance transdermal delivery of the Rspo agonist. In some embodiments, the applying comprises injecting the Rspo agonist subcutaneously. In some embodiments, the injecting is performed with one or more microneedles. In some embodiments, the injecting is performed with an array of microneedles. In some embodiments, the cosmetic composition is applied to the scalp of the subject. In some embodiments, the applying comprises contacting the scalp of the subject with a patch described herein. In some embodiments, the applying comprises applying the composition at least once a month. In some embodiments, applying comprises applying the composition at least once a week. In some embodiments, applying comprises applying the composition at least once a day.

The present invention provides cosmetic and pharmaceutical skin and hair care compositions for enhancing the growth and appearance of mammalian hair in an individual, particularly, although not exclusively, hair of the scalp. In some embodiments of the invention the cosmetic formulations comprise an effective dose of an Rspo agonist in a formulation that provides for penetration into the scalp. The formulations of the invention may further include pharmaceutically and/or cosmetically acceptable vehicle(s) and/or other skin and hair conditioning agents. Additional agents to enhance skin penetration may be included in the formulation.

In some embodiments, the methods and compositions of the invention relate to active Rspo agonists, including without limitation human Rspo2 protein and biologically active fragments and conjugates thereof, which are formulated in microneedles suitable for microinjection into the scalp, wherein the active Rspo2 is delivered to cells of the hair follicle, and thereby amplify endogenous Wnt signalling. The Rspo agonist compositions of the invention are provided in a dose that is effective to enhance Wnt signalling in hair follicles, and to thereby extend the anagen phase of the hair follicle growth cycle. A number of stem and progenitor cell populations within the hair follicle are Wnt responsive. Delivery of an Rspo agonist to these stem and progenitor populations maintains the cells in a self-renewing and proliferative state, enhancing the length and/or thickness of the hair shaft.

In some embodiments the Rspo agonist is a mammalian Rspo2 polypeptide, including without limitation human Rspo2 and active fragments thereof. In some embodiments, the Rspo2 polypeptide is provided in a topical formulation. In some embodiments the Rspo2 polypeptide is delivered in a microneedle formulation.

In the methods of the invention, a topical composition comprising an effective dose of an Rspo2 protein is administered to the scalp of an individual for a period of time sufficient to improve the appearance of the individual's hair. The individual may be a human suffering from androgenic alopecia. In some embodiments a topical composition comprising an effective dose of an Rspo2 protein is administered to the eyebrows or eyelashes of an individual for a period of time sufficient to improve the appearance of the eyebrows or eyelashes.

In some embodiments, a cosmetically acceptable skin care composition is provided, comprising an effective dose of a topically formulated Rspo2 polypeptide. The compositions of the invention can include a cosmetically acceptable vehicle to act as a diluent, dispersant or carrier for the active agents, so as to facilitate distribution and uptake when the composition is applied to the skin. Vehicles other than or in addition to water can include liquid or solid emollients, solvents, humectants, thickeners and powders. The cosmetically acceptable vehicle may range from 5% to 99.9%, preferably from 25% to 80% by weight of the composition, and can, in the absence of other cosmetic adjuncts, form the balance of the composition. The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. When the compositions of the invention are formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifier and coemulsifier may be present in the composition at a proportion ranging from 0.3% to 30% by weight, and preferably from 0.5% to 20% by weight, relative to the total weight of the composition.

These and other objects, advantages, and features of the invention will become apparent to those persons skilled in the art upon reading the details of the treatment methods, and in vitro and in vivo assay methods, as more fully described below.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1I. FIG. 1A illustrates how Wnt signaling fluctuates with the hair cycle. FIG. 1B illustrates Gomori trichrome staining of a tissue section through HFs in anagen. FIG. 1C Gomori trichrome staining of miniaturized telogen HFs (dotted yellow lines). Whole-mount Xgal staining of dorsal skin from an Axin2^{LacZ/+} mouse shows HFs in anagen (FIG. 1D) and HFs in telogen (FIG. 1E). FIG. 1F shows, immediately after shaving, asynchronous hair growth visualized on a mouse's back. FIG. 1G illustrates whole-mount Xgal staining of asynchronous HFs in the dorsal skin from an Axin2^{LacZ/+} mouse; the boxed area is shown in FIG. 1H at higher magnification. FIG. 1I illustrates qRT-PCR for Axin2 in 4 mm punch biopsies from the skin of 5 week-old mice in anagen, 9 week-old mice in telogen, and 12 week-old mice after wax-induced anagen. Abbreviations: a, anagen; t, telogen. Scale bars=500 μm; asterisk denotes P<0.05.

FIG. 2A-2F illustrate how Wnt signaling is elevated during anagen and lowered at the onset of catagen. FIG. 2A shows that waxing synchronizes the hair cycle, which has a duration of ˜21 days. FIG. 2B illustrates the localization of BrdU^{+ve} cells, FIG. 2C illustrates TUNEL activity, and FIG. 2D shows Lef1 immunostaining in tissue sections of waxed skin on the days indicated. FIG. 2E illustrates quantitative RT-PCR analysis of Axin2 and DKK1 expression in punch biopsies from waxed skin, collected on the days indicated. FIG. 2F shows qRT-PCR analysis of PCNA expression in 4 mm punch biopsies from waxed skin on the days indicated. Abbreviations: dp, dermal papilla; irs, inner root sheath; rs, root sheath; um, upper matrix; Im, lower matrix; se, sebaceous gland. Scale bar=50 μm; asterisk on BrdU^{+ve} tissue section indicates auto-fluorescent hair shaft; asterisk on graphs denotes P<0.05.

FIG. 3A-3D Illustrate how Rspo2 amplifies exogenous and endogenous Wnt signals. FIG. 3A illustrates that Rspo2 in combination with Wnt3a amplifies Wnt signaling ˜5-fold in mouse LSL cells. LSL cells, stably transfected with a Wnt-responsive luciferase reporter plasmid pSuperTOPFlash (Addgene) and a constitutive LacZ expression construct pEF/Myc/His/LacZ (Invitrogen) to normalize cell number, were treated with Rspo2 or Wnt3a, or the proteins in combination. The effect of Wnt3a concentration on Wnt signal amplification by Rspo2 in LSL cells is characterized. FIG. 3B illustrates that Rspo2 in combination with Wnt3a amplifies Wnt signaling 2.5-fold in mouse embryonic fibroblasts (MEFs). FIG. 3C illustrates an experiment where 250 ng of Rspo2 was delivered subcutaneously to 6-week old mice whose synchronized hair cycle was in anagen, or to 8-week old mice whose synchronized hair cycle was in telogen; after 24 h, qRT-PCR was used to analyze Axin2 expression. FIG. 3D illustrates that Rspo2 injections at PWD4 were sufficient to induce a 2-fold increase in Axin2 and Lef1 expression.

FIG. 4A-4L illustrate how Rspo2 prolongs anagen. FIG. 4A provides a schematic of experimental design. The backs of Axin2^{LacZ/+} mice were waxed and on post-wax day 4 (PWD 4), each mouse received four subcutaneous injections: two PBS (control) injections (2.5 μL) on one side and two Rspo2 injections (2.5 μL of a 100 ng/μL solution) on the contralateral side (N=5 mice). FIG. 4B illustrates that mice received daily injections from PWD 4-10. On PWD22 the skin was harvested and photographed. Brackets indicate Rspo2 injection sites where HFs can be visualized on the sub-dermal surface. PBS injection sites were indistinguishable from the remaining pelt. Dotted line indicates plane of section in C and D. FIG. 4C provides a representative whole-mount Xgal staining of an Axin2^{LacZ/+} pelt; bracket indicates one Rspo2 injection site; note surrounding HFs are in telogen. FIG. 4D is from the same pelt, the second Rspo2 injection site, and the intervening region. FIG. 4E corresponds to bracketed area in FIG. 4C; in the Rspo2 injection site, and provides Lef1 immunostaining of an anagen HF with corresponding Xgal staining. FIG. 4F corresponds to bracketed area in FIG. 4D; in the skin between the Rspo2 injection sites, Lef1 immunostaining of a telogen HF; Xgal as above. FIG. 4G illustrates the boxed region of FIG. 4E at higher magnification, showing Xgal^{+ve} hair follicles that are immunopositive for Lef1. FIG. 4H illustrates the boxed region of FIG. 4F at higher magnification, showing that hair follicles had progressed to the catagen phase and only the dermal papilla remained weakly positive for Lef1 immunostaining. FIG. 4I illustrates TUNEL staining of the upper and lower matrix of the hair follicle at the Rspo2 injection site. FIG. 4J illustrates TUNEL staining both in cells of the dermal papilla and the sebaceous gland. FIG. 4K illustrates Ki67 immunostaining in proliferating cells of the matrix and the inner root sheath. FIG. 4L illustrates few proliferating cells detectable in telogen hair follicles outside the Rspo2 injection site.

FIG. 5A-5K Illustrate how Rspo2 stimulates hair growth via activation of Lgr5^{+ve} stem cells in the HF. Xgal staining of HFs from Lgr5^{LacZ/+} mice, FIG. 5A on post-waxing day 1, in the ORS and DP; FIG. 5B on post-waxing day 7, in the ORS; FIG. 5C on post-waxing day 10 and FIG. 5D post-waxing day 14, ORS. FIG. 5E illustrates Lgr5 expression at times post waxing (in days). FIG. 5F illustrates that at the time points indicated (6-24 h), punch biopsies were collected from PBS injection sites (solid line) and Rspo2 injection site (dash-dot line); RNA was extracted and qRT-PCR was used to interrogate Lgr5 expression. Data are normalized were normalized to Lgr5 expression following intradermal PBS injections (solid line). FIG. 5G provides, from post-waxing day 22, representative whole-mount Xgal staining of an Lgr5^{LacZ/+} pelt from underneath and (FIG. 5H) on its side; dotted indicates Rspo2 injection site. FIG. 5H provides, from post-waxing day 22, Xgal stained HFs from an Lgr5^{LacZ/+} pelt from a non-injected site. FIG. 5I illustrates flat-mount Xgal stained telogen HFs from non-injected site; and FIG. 5J from an Rspo2 injection site. FIG. 5K illustrates effects after 10 days of PBS (N=5) or Rspo2 (N=5) injections, individual guard hair lengths and thicknesses (width) were measured using a 10× objective.

FIG. 6A-6E illustrate how human hair grows in response to a Wnt stimulus. Human hair follicles were harvested from the scalps of male volunteers and immediately placed into 24-well plates containing hair growth media, Rspo2 (2 ng/μL) or WNT3A (0.15 ng/μL)+Rspo2 (2 ng/μL). The number of hair follicles evaluated are indicated. FIG. 6A shows BrdU incorporation, indicating ongoing cell division in culture, by cultured hair follicles in a cross section containing the dermal papilla, inner root sheath, and matrix. FIG. 6B shows BrdU incorporation in a cross-section of a hair follicle showing the outer root sheath. FIG. 6C-6D illustrates over a 14-day time period, human hair follicles treated with WNT3A+Rspo2 show significant growth. FIG. 6C illustrates representative hair follicles treated with media show relative growth of hair follicles incubated in media. FIG. 6D illustrates the relative growth rate of hair follicles incubated in media (dash-dot line), Rspo2 (dash line) or WNT3A+Rspo2 (solid line). Only hair follicles incubated in WNT3A+Rspo2 show significantly greater growth. FIG. 6E shows that, although the overall growth rate of human hair follicles is not improved by Rspo2 treatment, their survival in vitro is transiently prolonged. By day 14, hair follicles have ceased growing.

### DEFINITIONS

Before the present methods are described, it is to be understood that this invention is not limited to particular methods described, as such can, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges can independently be included in the smaller ranges encompassed within the invention, subject to any specifically excluded limit in the stated range. Also used herein, ranges and amounts can be expressed as “about” a particular value or range. About also includes the exact amount. Hence “about 5 μL” means “about 5 μL” and also “5 μL.” Generally, the term “about” includes an amount that would be expected to be within ±10% of the amount.

Unless defined otherwise, all technical and scientific terms used herein have the same meaning as commonly understood by one of ordinary skill in the art to which this invention belongs. Singleton et al, Dictionary of Microbiology and Molecular Biology 2nd ed., J. Wiley & Sons (New York, N.Y. 1994), provides one skilled in the art with a general guide to many of the terms used in the present application. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

“Mammalian hair,” as used herein, includes hair on any part of the body of a mammal and may include facial, cranial, or body hair. Of particular interest for increased hair growth is the hair present on the human scalp. Hair of the eyelashes and eyebrows is also of interest for growth modification with the methods of the invention.

“Regulating hair growth,” namely mammalian hair growth, includes reducing, modulating, inhibiting, attenuating, retarding, promoting, enhancing, and/or the diminution of hair growth, and/or reducing shaving frequency. Enhancement or promotion of hair growth is of particular interest.

“NL (Neogenic-Like) follicular structure” includes unattached primitive follicular structure, with only one of the following “small” traits: shaft, sebaceous gland, or pore. Dermal channel is absent or inconclusive. Further subcategories of NL include: NL with DP (dermal papilla)/active, NL with DP/inactive, NL without DP/active, and NL without DP/inactive.

“Nonvellus hair” includes “terminal hair.”

“PEL (Pre-Existing-Like) follicular structure” includes an unattached primitive follicular structure, with one or more of the following “large” traits or two or more of the following “small” traits: shaft (large or small), sebaceous gland (large or small), or pore (large or small). Dermal channel is present. Further subcategories of PEL include: PEL with DP (dermal papilla)/active, PEL with DP/inactive, PEL without DP/active, and PEL without DP/inactive.

“PELA (Pre-Existing-Like, Attached) follicular structure” includes primitive follicular structure that is attached to larger, mature, pilosebaceous unit that extends to the epidermis.

“Terminal hair” includes large, usually pigmented hairs on scalp and body. Hair shaft diameters are typically 30 μm or greater.

“Promoting hair growth” includes stimulating an increase in total hair mass and/or length. Such increase includes increased length and/or growth rate of hair shafts (i.e. follicles), increased number of hairs, and/or increased hair thickness. Some or all of the above end results can be achieved by prolonging or activating anagen, the growth phase of the hair cycle, or by shortening or delaying the catagen and telogen phases. “Promoting hair growth” may include preventing, arresting, decreasing, delaying and/or reversing hair loss.

“Anagen,” as used herein, refers to the active growth phase of hair follicles. In the anagen phase, cells in the root of the hair divide rapidly, adding to the hair shaft. During this phase, the hair grows about 1 cm every 28 days. Scalp hairs stay in this active phase of growth for 2-6 years.

“Catagen,” as used herein, refers to the hair growth phase that occurs at the end of the anagen phase. It signals the end of the active growth of a hair. This phase lasts for about 2-3 weeks while a club hair is formed.

“Telogen,” as used herein, refers to the resting phase of the hair follicle. At any given time, 10%-15% of all hairs are in the telogen phase. This phase lasts for about 100 days for hairs on the scalp and much longer for hairs on the eyebrows, eyelashes, arms and legs. During this phase, the hair follicle is completely at rest and the club hair is completely formed. Pulling out a hair in this phase will reveal a solid, hard, dry, white material at the root. About 25-100 telogen hairs are shed normally each day.

A “growth state” of a cell refers to the rate of proliferation of the cell and/or the state of differentiation of the cell.

An “active agent” is an agent, drug, compound, or composition of matter or mixture thereof which provides some pharmacologic, often beneficial, effect.

“Topical application” or “topical,” as used herein, means to apply or spread the compositions of the present invention onto the surface of a keratinous tissue.

“Dermatologically-acceptable,” as used herein, means that the compositions or components thereof so described are suitable for use in contact with mammalian keratinous tissue without undue toxicity, incompatibility, instability, allergic response, and the like.

“Safe and effective amount” as used herein, means an amount of a compound or composition sufficient to significantly induce a positive benefit, preferably a hair growth regulating benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “R-spondin agonist” specifically includes proteins R-spondin 1, R-spondin 2, R-spondin 3 and R-spondin 4, e.g., the native human proteins, or protein derived from a mammal of interest, such as by truncation of the proteins, fusion or chimeras of the native proteins, e.g., fusion products with a portion of an immunoglobulin; pegylated version of the native protein, in addition to antibodies and other mimetics that provide for the biological activity of an R-spondin.

A “native sequence” polypeptide is one that has the same amino acid sequence as a polypeptide derived from nature. Such native sequence polypeptides can be isolated from cells producing endogenous protein, e.g., Rspo2, or can be produced by recombinant or synthetic means. Thus, a native sequence polypeptide can have the amino acid sequence of, e.g., naturally occurring human polypeptide, murine polypeptide, or polypeptide from any other mammalian species, or from non-mammalian species, e.g. Drosophila, C. elegans, or the like. In some instances, the “native sequence” polypeptide also includes the N-terminal methionine. In some instances, the “native sequence” Rspondin polypeptide does not include the N-terminal methionine. In certain embodiments, the compositions of the disclosure do not include a native sequence polypeptide of a Rspondin polypeptide, for example the composition does not include one or more of Rspo1, Rspo2, Rspo3 and Rspo4.

A “variant” polypeptide means a functionally active polypeptide as defined below having less than 100% sequence identity with a native sequence polypeptide. Such variants include longer or shorter polypeptides than the native sequence polypeptide, for example wherein one or more amino acid residues are added at the N- or C terminus of, or within, the native sequence; or from about one to 300 amino acid residues are deleted from the native sequence. Variant polypeptides include polypeptides with one or more amino acid substitutions in comparison to the native polypeptide sequence, or derivatives of a polypeptide sequence, wherein an amino acid residue has been covalently modified so that the resulting product has a non-naturally occurring amino acid.

According to the present invention “formulation” is a generic term which can encompass additives, personal care, cosmetic, and dermopharmaceutical compositions, and additives and personal care, cosmetic, and dermopharmaceutical compositions. Formulations can also cover additives and personal care, cosmetic and dermopharmaceutical compositions comprising an active agent.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity can be determined, for example, by comparing sequence information using the advanced BLAST computer program, including version 2.2.9 with default parameters, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 70% to 100% and integer values therebetween. In general, an exact match indicates 100% identity over the length of the shortest of the sequences being compared (or over the length of both sequences, if identical).

A “functionally active” Rspo polypeptide (e.g. Rspo2 polypeptide) retains the effector functions that are directly or indirectly caused or performed by native sequence polypeptides. Effector functions of native sequence Rspo polypeptides include enhancement of Wnt activity.

The term “amino acid” refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, a-amino acids, natural amino acids, non-natural amino acids, and amino acid analogs. The term “α-amino acid” refers to a molecule containing both an amino group and a carboxyl group bound to a carbon which is designated the α-carbon. The term “amino acid” refers to a molecule containing both an amino group and a carboxyl group in a β configuration. The term “naturally occurring amino acid” refers to any one of the twenty amino acids commonly found in peptides synthesized in nature.

### DETAILED DESCRIPTION OF THE EMBODIMENTS

Compositions are provided for enhancing the growth of hair, where the composition comprises an Rspo agonist, including particularly mammalian R-spondin polypeptides, e.g. Rspo1, Rspo2, Rspo3,and Rspo4, at a dose effective to promote mammalian hair growth, which may be liposomally formulated to enhance penetration into hair follicles.

The present invention provides cosmetic skin and hair care compositions for enhancing the growth and appearance of mammalian hair in an individual, particularly hair of the scalp. In some embodiments of the invention the cosmetic formulations are topical formulations. In some embodiments of the invention the cosmetic formulations are injectable, including microinjectable. In some embodiments the cosmetic formulations comprise a dose of an Rspo agonist effective to promote anagen phase of the hair cycle, resulting in more rapid hair growth. The formulations of the invention may further include cosmetically acceptable vehicle(s) and/or other skin and hair conditioning agents. Additional agents to enhance skin penetration may be included in the formulation.

R-spondins (RSPOs), such as Rspo2, are secreted proteins that regulate beta-catenin through the control of the internalization of Frizzled and LRP5/6 receptors. Like other members of the Rspo family (e.g., Rspo1, Rspo3, and Rspo4), the 243-amino acid human Rspo2 protein contains an N-terminal signal peptide, 2 furin-like domains, a thrombospondin type-1 domain, and a C-terminal low-complexity region enriched with positively charged amino acids. Rspo2 expression has been detected in organs of endodermal origin, including colon, rectum, small intestine, and lung, with decreased expression in corresponding tumors. Rspo2 functions in a positive feedback loop to modulate the WNT/beta-catenin cascade. The human Rspo2 gene contains 6 coding exons, and maps to chromosome 8q23.1. The genetic reference sequence for R-spondin 2 may be accessed at Genbank, locus NM_178565, and as described by Clark et al. (2003) Genome Res. 13 (10), 2265-2270, herein specifically incorporated by reference, and exemplary Rspo2 protein sequences are provided herein as SEQ ID NO:1 and 2 (isotype 1 and 2, respectively).

“R-spondin1” protein is described in Genbank Accession NP_001033722. R-spondin1 (Rspo1) is one of the four proteins in the R-spondin protein family (Four human paralogs of R-spondin include R-spondin1-4). Rspo1 is a secreted glycoprotein containing a leading signal peptide, two cysteine-rich, furin-like domains, and one thrombospondin type 1 domain.

Rspo1 and Rspo2 have no homology with Wnts, but modulate Wnts to enhance β-catenin-dependent signaling.

Included in the R-spondin molecules of interest, e.g. R-spondin 1, 2, 3, and 4, are “chimeric” polypeptides comprising an R-spondin polypeptide or portion thereof, e.g., one or more domains, fused or bonded to a heterologous polypeptide. The chimeric polypeptide will generally share at least one biological property in common with a native R-spondin polypeptide. Examples of chimeric polypeptides include immunoadhesins, which combine a portion of the native polypeptide with an immunoglobulin sequence, particularly an Fc region of an immunoglobulin, which molecules are known in the art to provide for improved pharmacokinetic properties. For example, a commercially available product is the full-length human R-spondin1 fused at its C-terminus to the Fc domain of human IgG1. This fusion increases the stability of the protein in vitro and in vivo without compromising its biological activity. See de Lau, et al (2011) Nature 476: 293; Carmon et al. (2011) PNAS 108: 11452, each herein specifically incorporated by reference.

Where the agent is an R-spondin agonist, such as a R-spondin polypeptide, fusion thereof (e.g. R-spondin2-Fc), truncation product thereof, fragment thereof, or any combination thereof, and any other chimeric protein of any combination of sequences derived from Rspo1, Rspo2, Rspo3 and Rspo4, for topical or subcutaneous administration, the effective dose may be at least about 0.01 μg/dose, at least about 0.05 μg/dose, at least about 0.1 μg/dose, at least about 0.25 μg/dose, at least about 0.5 μg/dose, at least about 1 μg/dose, at least about 2 μg/dose, at least about 2.5 μg/dose, at least about 5 μg/dose, at least about 10 μg/dose, at least about 50 μg/dose, at least about 100 μg/dose, at least about 250 μg/dose, at least about 500 μg/dose, at least about 750 μg/dose, at least about 1 mg/dose, at least about 10 mg/dose, at least about 50 mg/dose, or at least about 100 mg/dose. In certain embodiments, the dose of R-spondin agonist may be administered one time per day, two times per day, three times per day, four times per day or even five times per day. In certain embodiments, the dose of R-spondin agonist is administered at least one time every other day, at least one time every third day, at least one time every fourth day, at least one time every fifth day, at least one time every sixth day or at least one time every seventh day.

Where the agent is an R-spondin agonist for topical or subcutaneous administration the effective dose may be at most about 1 μg/dose, at most about 2 μg/dose, at most about 2.5 μg/dose, at most about 5 μg/dose, at most about 10 μg/dose, at most about 50 μg/dose, at most about 100 μg/dose, at most about 250 μg/dose, at most about 500 μg/dose, at most about 750 μg/dose, at most about 1 mg/dose, at most about 10 mg/dose, at most about 50 mg/dose, at most about 100 mg/dose, at most about 250 mg/dose, at most about 500 mg/dose, at most about 750 mg/dose, or at most about 1 g/dose. In certain embodiments, any one values discussed in the preceding paragraph may be combined with the values of the current paragraph to produce a range envisioned by the disclosure. For example, an effective dose of at least about 0.01 μg/dose to at most about 10 μg/dose of a R-spondin agonist is administered. In some embodiments, an effective dose of R-spondin agonist is at least 1 ng, at least 5 ng, at least 10 ng, at least 20 ng, at least 30 ng, at last 40 ng, at least 50 ng, at least 75 ng, at least 100 ng, at least 125 ng, at least 150 ng, at least 175 ng, at least 200 ng, at least 250 ng, at least 500 ng, or at least 1 mg. In some embodiments, an effective dose is at most 1 ng, at most 5 ng, at most 10 ng, at most 20 ng, at most 30 ng, at last 40 ng, at most 50 ng, at most 75 ng, at most 100 ng, at most 125 ng, at most 150 ng, at most 175 ng, at most 200 ng, at most 250 ng, at most 500 ng, or at most 1 mg. In some embodiments, an effective dose is from about 5 ng to about 250 ng, from about 10 ng to about 200 ng, or from about 50 ng to about 100 ng.

In certain embodiments, one dose of R-spondin agonist may be administered at two or more different locations. For example a dose of 100 ng may be administered at two or more locations on the scalp either topically or subcutaneously, such that the total dose administered at the two or more locations adds up to a total of 100 ng . In certain embodiments, the R-spondin agonist is administered to a surface area of the scalp, such as through the use of a one or more patches or microarrays of needles in contact with said surface area. The surface area of the scalp may be a surface of from about 1 cm² to about 100 cm², such as about 5 cm² to about 50 cm². The patch may provide a total topical dose of R-spondin agonist to said surface area, wherein said dose is evenly administered or substantially evenly administered over said surface area. The microarray may provide a total dose of R-spondin agonist to the subcutaneous area below said surface area, wherein said dose is evenly administered or substantially evenly administered over said surface area.

The final concentration of an R-spondin agonist in a formulation of the disclosure may be at least about 0.1 mg/mL, at least about 0.5 mg/mL, at least about 1 mg/mL, at least about 5 mg/mL, at least about 10 mg/mL, at least about 50 mg/mL, at least about 100 mg/mL, at least about 500 mg/mL, or more.

The final concentration of an R-spondin agonist in a formulation of the disclosure may be at most about 0.1 mg/mL, at most about 0.5 mg/mL, at most about 1 mg/mL, at most about 5 mg/mL, at most about 10 mg/mL, at most about 50 mg/mL, at most about 100 mg/mL, at most about 500 mg/mL, or more. In certain embodiments, any one values discussed in the preceding paragraph may be combined with the values of the current paragraph to produce a range envisioned by the disclosure. For example, the final concentration of an R-spondin agonist in the formulations of the disclosure may be at least about 0.1 mg/mL to at most about 10 mg/mL. In some embodiments, the final concentration of R-spondin agonist in a formulation of the disclosure may be at least 100 ng/mL, at least 250 ng/mL, at least 500 ng/mL, at least 1 μg/mL, at least 5 μg/mL, at least 10 μg/mL, at least 20 μg/mL, at least 30 μg/mL, at least 40 μg/mL, at least 50 μg/mL, at least 60 μg/mL, at least 70 μg/mL, at least 80 μg/mL, at least 90 μg/mL, or at least 100 μg/mL. In some embodiments, the final concentration of R-spondin agonist in a formulation of the disclosure may be at most 100 ng/mL, at most 250 ng/mL, at most 500 ng/mL, at most 1 μg/mL, at most 5 μg/mL, at most 10 μg/mL, at most 20 μg/mL, at most 30 μg/mL, at most 40 μg/mL, at most 50 μg/mL, at most 60 μg/mL, at most 70 μg/mL, at most 80 μg/mL, at most 90 μg/mL, or at most 100 μg/mL. In some embodiments, the final concentration is from about 1 μg/mL to about 50 μg/mL or from about 10 μg/mL to about 40 μg/mL.

In some embodiments, a given dosing schedule comprising one or more administrations of a R-spondin agonist may be repeated on a daily, weekly, biweekly, triweekly, monthly, bimonthly, annually, semi-annually, or any other period as may be determined by a medical professional. A repeated dosing schedule may be repeated for a fixed period of time determined at the start of the schedule; may be terminated, extended, or otherwise adjusted based on a measure of therapeutic effect, such as a level of reduction in the presence of detectable hair loss (e.g. a reduction of at least 50%, 60%, 70%, 80%, 90%, 95%, 99%, or 100%); or may be terminated, extended, or otherwise adjusted for any other reason as determined by a medical professional.

A R-spondin agonist disclosed herein can be administered as part of a combination treatment, wherein the R-spondin agonist may be administered with one or more additional therapeutic agents. Such one or more additional agents can be administered simultaneously or separately with respect to the R-spondin agonist. Administration in combination utilizing one or more additional agents includes, for example, simultaneous administration of two agents in the same dosage form, simultaneous administration in separate dosage forms, and separate administration. For example, multiple therapeutic agents can be formulated together in the same dosage form and administered simultaneously. Alternatively multiple therapeutic agents can be simultaneously administered, wherein the agents are present in separate formulations.

In another alternative, an R-spondin agonist of the present invention can be administered followed by one or more additional agents, or the R-spondin agonist of the present invention can be administered preceded by one or more additional agents. In the separate administration protocol, a R-spondin agonist of the present invention and one or more additional agents may be administered a few minutes apart, or a few hours apart, or a few days apart. The term “combination treatments” also embraces the administration of the polypeptides as described herein in further combination with other biologically active compounds or ingredients and non-drug therapies.

Methods of determining the most effective means and dosage of administration are well known to those of skill in the art and will vary with the composition used for therapy, the purpose of the therapy, the target tissue being treated, and the subject being treated. Single or multiple administrations (e.g. about or more than about 1, 2, 3, 4, 5, 6, 7, 8, 9, 10, 11, 12, 13, 14, 15, 16, 17, 18, 19, 20, 25, 30, or more doses) can be carried out with the dose level and pattern being selected by the treating physician.

Dosages for the R-spondin agonist may be determined empirically in individuals who have been given one or more administrations of a R-spondin agonist. Individuals may be given incremental doses of a R-spondin agonist and to assess efficacy of an R-spondin agonist, further hair loss can be monitored.

Administration of an R-spondin agonist according to the methods of the invention can be continuous or intermittent, depending, for example, on the recipient's physiological condition, whether the purpose of the administration is therapeutic or prophylactic, and other factors known to skilled practitioners.

In some embodiments, a R-spondin agonist and/or any additional therapeutic compound of the invention is administered in multiple doses. Dosing may be about once, twice, three times, four times, five times, six times, or more than six times per day. Dosing may be about once a month, once every two weeks, once a week, or once every other day.

Administration of the R-spondin agonist of the invention may continue as long as necessary. In some embodiments, an R-spondin agonist of the invention is administered for more than 1, 2, 3, 4, 5, 6, 7, 14, or 28 days. In some embodiments, an R-spondin agonist of the invention is administered for less than 28, 14, 7, 6, 5, 4, 3, 2, or 1 day. In some embodiments, an R-spondin agonist of the invention is administered chronically on an ongoing basis, e.g., for the treatment of chronic effects.

When a combination treatment of the invention is administered as a composition that comprises one or more agents, and one agent has a shorter half-life than another agent, the unit dose forms may be adjusted accordingly.

In some instances, a functionally active Rspo variant will have an amino acid sequence that has at least about 30%, at least about 40%, at least about 50%, at least about 60%, at least about 70%, at least about 80%, at least about 90%, at least about 95%, or at least about 99% amino acid sequence identity with a native sequence Rspo polypeptide. In one embodiment the native sequence Rspo polypeptide is a mammalian (e.g., human) Rspo polypeptide such as R-spondin2. In one embodiment, the Rspo agonist is a Rspo polypeptide selected from the group consisting of R-spondin1, R-spondin2, R-spondin3, and R-spondin4. In one embodiment, the Rspo polypeptide is human R-spondin1. In one embodiment, the Rspo polypeptide is human R-spondin2. In one embodiment, the Rspo polypeptide is human R-spondin3. In one embodiment, the Rspo polypeptide is human R-spondin4.

In some instances, an Rspo polypeptide can be truncated. In some cases, the Rspo polypeptide comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% the length of an Rspo polypeptide. In one embodiment, the Rspo polypeptide is a human Rspo polypeptide. In one embodiment, the Rspo polypeptide is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% the length of human R-spondin (e.g., R-spondin1, R-spondin2, R-spondin3, and R-spondin4).

In some cases, the Rspo polypeptide comprises at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99% the length of an Rspo polypeptide. In one embodiment, the Rspo polypeptide is a human Rspo polypeptide. In one embodiment, the Rspo polypeptide is at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98%, or at most 99% the length of human R-spondin (e.g., R-spondin1, R-spondin2, R-spondin3, and R-spondin4).

In some cases, the Rspo polypeptide is between 10% and 99%, between 10% and 90%, between 20% and 90%, between 30% and 80%, between 40% and 60%, or between 40% and 80% the length of human R-spondin (e.g., R-spondin1, R-spondin2, R-spondin3, and R-spondin4).

In some instances, the Rspo agonist is a polypeptide comprising a sequence identical to at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, or at least 120 amino acids of SEQ ID NO: 1 or SEQ ID NO: 2. For example, the Rspo agonist can comprise a sequence identical to at least 70 consecutive amino acids of the native sequence of an Rspo polypeptide.

In some instances, the Rspo agonist is a polypeptide comprising a sequence identical to at most 10 amino acids, at most 20 amino acids, at most 30 amino acids, at most 40 amino acids, at most 50 amino acids, at most 60 amino acids, at most 70 amino acids, at most 80 amino acids, at most 90 amino acids, at most 100 amino acids, at most 110 amino acids, or at most 120 amino acids of SEQ ID NO: 1 or SEQ ID NO: 2. For example, the Rspo agonist can comprise a sequence identical to at most 70 consecutive amino acids of the native sequence of an Rspo polypeptide.

In some instances, the Rspo agonist is a polypeptide identical to at most 10 amino acids, at most 20 amino acids, at most 30 amino acids, at most 40 amino acids, at most 50 amino acids, at most 60 amino acids, at most 70 amino acids, at most 80 amino acids, at most 90 amino acids, at most 100 amino acids, at most 110 amino acids, or at most 120 amino acids of SEQ ID NO: 1 or SEQ ID NO: 2. For example, the Rspo agonist can be a sequence identical to 70 consecutive amino acids of the native sequence of an Rspo polypeptide.

In some instances, two or more Rspo polypeptides and/or truncated Rspo polypeptides can be administered. The two or more Rspo polypeptides and/or truncated Rspo polypeptides can be administered simultaneously. The two or more Rspo polypeptides and/or truncated Rspo polypeptides can be administered sequentially. The two or more Rspo polypeptides and/or truncated Rspo polypeptides can be administered in the same or in different concentrations. For example, R-spondinl can be truncated and a minimal combination of short polypeptides derived from the intact polypeptide can be determined that are sufficient to induce at least a portion of the effect of the whole protein. Non-limiting examples are short polypeptides derived from R-spondinl are MQFRLFSFAL, IILNCMDYSH, CQGNRWRRSK, RASYVSNPIC, KGCLSCSKDN, GCSRCQQKLF, FFLRREGMRQ, YGECLHSCPS, GYYGHRAPDM, NRCARCRIEN, CDSCFSKDFC, TKCKVGFYLH, RGRCFDECPD, GFAPLEETME, CVEGCEVGHW, SEWGTCSRNN, RTCGFKWGLE, TRTRQIVKKP, VKDTILCPTI, AESRRCKMTM, RHCPGGKRTP, KAKEKRNKKK, KRKLIERAQE, and QHSVFLATDR.

### Delivery Methods

Various methods known in the art can be used for the delivery of R-spondin to hair follicles, including patches, chemical enhancers, ultrasound, electroporation, cavitational ultrasound, microneedles, thermal ablation, microdermabrasion, and the like.

In some embodiments of the invention, an effective dose of an R-spondin agonist is injected into the skin (e.g., the scalp). In one embodiment, the injection device is a needle and syringe. In some embodiments, the injection device is a microneedle. Useful devices for this purpose include, without limitation, microneedle and microneedle array devices. A microneedle device is typically applied to skin. The stratum corneum is the outer layer, generally between 10 and 50 cells, or between 10 and 20 μm thick. Unlike other tissue in the body, the stratum corneum contains “cells” (called keratinocytes) filled with bundles of cross-linked keratin and keratohyalin surrounded by an extracellular matrix of lipids. It is this structure that is believed to give skin its barrier properties, which prevents therapeutic transdermal administration of many drugs. Below the stratum corneum is the viable epidermis, which is between 50 and 100 μm thick. The viable epidermis contains no blood vessels, and it exchanges metabolites by diffusion to and from the dermis. Beneath the viable epidermis is the dermis, which is between 1 and 3 mm thick and contains blood vessels, lymphatics, and nerves.

A microneedle device may include a substrate; one or more microneedles; and, optionally, a reservoir for delivery of an R-spondin agonist, as well as pump(s), sensor(s), and/or microprocessor(s) to control the interaction of the foregoing. The substrate of the device can be constructed from a variety of materials, including metals, ceramics, semiconductors, organics, polymers, and composites. The substrate includes the base to which the microneedles are attached or integrally formed. A reservoir may also be attached to the substrate.

The microneedles of the device can be constructed from a variety of materials, including metals, ceramics, semiconductors, organics, polymers, and composites. Preferred materials of construction include pharmaceutical grade stainless steel, gold, titanium, nickel, iron, gold, tin, chromium, copper, alloys of these or other metals, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative nonbiodegradable polymers include polycarbonate, polymethacrylic acid, ethylenevinyl acetate, polytetrafluorethylene and polyesters.

Polymeric microneedles (MNs) can provide biocompatibility, biodegradability, strength, toughness, and optical clarity. To accurately produce the micro-scale dimensions of polymer MNs, a variety of mould-based techniques, such as casting, hot embossing, injection molding, and investment molding may be used, e.g. beveled-tip, chisel-tip, and tapered-cone polydimethylsiloxane (PDMS) molds. Polymeric materials of interest for fabrication include without limitation; poly (methylmetha-acrylate) (PMMA), poly-L-lactic acid (PLA), poly-glycolic acid (PGA), and poly-lactic-co-glycolic acid (PLGA), cyclic-olefin copolymer, poly (vinyl pyrrolidone), and sodium carboxymethyl cellulose. Sugars have also been used to fabricate MNs, such as galactose, maltose, aliginate, chitosan, and dextrin. Materials may be cross-linked through ion exchange, photo-polymerization, and the like, and the active agent may be embedded in a dissolving microneedle.

Generally, the microneedles should have the mechanical strength to remain intact for delivery of the active agent while being inserted into the skin, and while being removed. In certain embodiments, the microneedles are formed of biodegradable polymers, which may be left in the skin to degrade, and for these the mechanical requirement may be less stringent.

The microneedles can be formed of a porous solid, with or without a sealed coating or exterior portion, or hollow. As used herein, the term “porous” means having pores or voids throughout at least a portion of the microneedle structure, sufficiently large and sufficiently interconnected to permit passage of fluid and/or solid materials through the microneedle. As used herein, the term “hollow” means having one or more substantially annular bores or channels through the interior of the microneedle structure, having a diameter sufficiently large to permit passage of fluid and/or solid materials through the microneedle. The annular bores may extend throughout all or a portion of the needle in the direction of the tip to the base, extending parallel to the direction of the needle or branching or exiting at a side of the needle, as appropriate. A solid or porous microneedle can be hollow. One of skill in the art can select the appropriate porosity and/or bore features required for specific applications. For example, one can adjust the pore size or bore diameter to permit passage of the particular material to be transported through the microneedle device.

The microneedles can have straight or tapered shafts. A hollow microneedle that has a substantially uniform diameter, which needle does not taper to a point, is referred to herein as a “microtube.” As used herein, the term “microneedle” includes, although is not limited to both microtubes and tapered needles unless otherwise indicated. In a preferred embodiment, the diameter of the microneedle is greatest at the base end of the microneedle and tapers to a point at the end distal the base. The microneedle can also be fabricated to have a shaft that includes both a straight (untapered) portion and a tapered portion.

The microneedles can be formed with shafts that have a circular cross-section in the perpendicular, or the cross-section can be non-circular. For example, the cross-section of the microneedle can be polygonal (e.g. star-shaped, square, triangular), oblong, or another shape. The shaft can have one or more bores. The cross-sectional dimensions typically are between about 10 nm and 1 mm, preferably between 1 micron and 200 microns, and more preferably between 10 and 100 μm. The outer diameter is typically between about 10 μm and about 100 μm, and the inner diameter is typically between about 3 μm and about 80 μm.

The length of the microneedles typically is between about 1 micron and 1 mm, preferably between 10 microns and 500 microns, and more preferably between 30 and 200 μm. The length is selected for the particular application, accounting for both an inserted and uninserted portion. An array of microneedles can include a mixture of microneedles having, for example, various lengths, outer diameters, inner diameters, cross-sectional shapes, and spacings between the microneedles.

The microneedles can be oriented perpendicular or at an angle to the substrate. Preferably, the microneedles are oriented perpendicular to the substrate so that a larger density of microneedles per unit area of substrate can be provided. An array of microneedles can include a mixture of microneedle orientations, heights, or other parameters.

In some embodiments, the substrate and/or microneedles, as well as other components, are formed from flexible materials to allow the device to fit the contours of the skin to which the device is applied. A flexible device will facilitate more consistent penetration during use, since penetration can be limited by deviations in the attachment surface.

The microneedle device may include a reservoir in communication with the microneedles. The reservoir can be attached to the substrate by any suitable means. In a preferred embodiment, the reservoir is attached to the back of the substrate (opposite the microneedles) around the periphery, using an adhesive agent (e.g., glue). A gasket may also be used to facilitate formation of a fluid-tight seal.

The microneedle device can include one or a plurality of chambers for storing materials to be delivered. In the embodiment having multiple chambers, each can be in fluid connection with all or a portion of the microneedles of the device array. In one embodiment, at least two chambers are used to separately contain an Rspo agonist and an administration vehicle (e.g., saline) in order to prevent or minimize degradation during storage. Immediately before use, the contents of the chambers are mixed. Mixing can be triggered by any means, including, for example, mechanical disruption (i.e. puncturing or breaking), changing the porosity, or electrochemical degradation of the walls or membranes separating the chambers.

The microneedle device also must be capable of transporting peptides or proteins across the skin at a rate sufficient to be therapeutically useful. The device may include a housing with microelectronics and other micromachined structures to control the rate of delivery either according to a preprogrammed schedule or through active interface with the patient, a healthcare professional, or a biosensor. The rate can be controlled by manipulating a variety of factors, including the characteristics of the formulation to be delivered (e.g., its viscosity, electric charge, and chemical composition); the dimensions of each microneedle (e.g., its outer diameter and the area of porous or hollow openings); the number of microneedles in the device; the application of a driving force (e.g., a concentration gradient, a voltage gradient, a pressure gradient); and the use of a valve.

The rate also can be controlled by interposing between the drug in the reservoir and the opening(s) at the base end of the microneedle polymeric or other materials selected for their diffusion characteristics. For example, the material composition and layer thickness can be manipulated using methods known in the art to vary the rate of diffusion of the drug of interest through the material, thereby controlling the rate at which the drug flows from the reservoir through the microneedle and into the tissue.

Transportation of proteins through the microneedles can be controlled or monitored using, for example, various combinations of valves, pumps, sensors, actuators, and microprocessors. These components can be produced using standard manufacturing or microfabrication techniques. Actuators that may be useful with the microneedle devices disclosed herein include micropumps, microvalves, and positioners.

Flow of molecules through the microneedles can occur based on diffusion, capillary action, or can be induced using conventional mechanical pumps or nonmechanical driving forces, such as electroosmosis or electrophoresis, or convection. For example, in electroosmosis, electrodes are positioned on the skin, one or more microneedles, and/or the substrate adjacent the needles, to create a convective flow which carries oppositely charged ionic species and/or neutral molecules toward or into the biological barrier. The microneedle device may be used in combination with another mechanism that enhances the permeability of the skin, for example by increasing cell uptake or membrane disruption, using electric fields, ultrasound, chemical enhancers, viruses, pH, heat and/or light.

The flow of peptides and proteins can be regulated using a wide range of valves or gates. These valves can be the type that are selectively and repeatedly opened and closed, or they can be single-use types. For example, in a disposable, single-use drug delivery device, a fracturable barrier or one-way gate may be installed in the device between the reservoir and the opening of the microneedles. When ready to use, the barrier can be broken or gate opened to permit flow through the microneedles. Other valves or gates used in the microneedle devices can be activated thermally, electrochemically, mechanically, or magnetically to selectively initiate, modulate, or stop the flow of molecules through the needles. In a preferred embodiment, flow is controlled by using a rate-limiting membrane as a “valve.”

Useful sensors may include sensors of pressure, temperature, chemicals, and/or electromagnetic fields. Biosensors can be employed, and in one arrangement, are located on the microneedle surface, inside a hollow or porous microneedle, or inside a device in communication with the body tissue via the microneedle (solid, hollow, or porous). These microneedle biosensors may include any suitable transducers, including but not limited to potentiometric, amperometric, optical, magnetic and physiochemical.

In some embodiments, transdermal patches store the active agent in a reservoir that is enclosed on one side with an impermeable backing and has an adhesive that contacts the skin on the other side. Some designs employ drug dissolved in a liquid or gel-based reservoir, which can simplify formulations and permit the use of liquid chemical enhancers, such as ethanol. These designs characteristically are composed of four layers: an impermeable backing membrane; a drug reservoir; a semi-permeable membrane that may serve as a rate-limiting barrier; and an adhesive layer. Other designs incorporate the drug into a solid polymer matrix, which simplifies manufacturing. Matrix systems can have three layers, by eliminating the semi-permeable membrane, or just two layers, by incorporating the drug directly into the adhesive. A variation on the transdermal patch applies a metered liquid spray, gel or other formulation to the skin that, upon evaporation or absorption, can drive drugs into the stratum corneum, which in turn serves as the drug reservoir for extended release into the viable epidermis over hours.

Transdermal delivery systems may comprise a skin permeability enhancer. Enhancement methods with patches include conventional chemical enhancers, iontophoresis and non-cavitational ultrasound. Many effective chemical enhancers disrupt the highly ordered bilayer structures of the intracellular lipids found in stratum corneum by inserting amphiphilic molecules into these bilayers to disorganize molecular packing or by extracting lipids using solvents and surfactants to create lipid packing defects of nanometer dimensions. Hundreds of different chemical enhancers have been studied, including off-the-shelf compounds and others specifically designed and synthesized for this purpose, such as Azone (1-dodecylazacycloheptan-2-one) and SEPA (2-n-nonyl-1,3dioxolane).

Liposomes, dendrimers and microemulsions have also been used as chemical enhancers with supramolecular structure that can not only increase skin permeability, but also increase drug solubilization in the formulation and drug partitioning into the skin. Their supramolecular size generally precludes penetration into the skin and thereby helps localize effects to the stratum corneum.

Iontophoresis typically applies a continuous low-voltage current, providing an electrical driving force for transport across stratum corneum. Charged drugs are moved via electrophoresis, while weakly charged and uncharged compounds can be moved by electroosmotic flow of water generated by the preferential movement of mobile cations (e.g., Na⁺) instead of fixed anions (e.g., keratin) in the stratum corneum. Current applications emphasize the ability of iontophoresis to provide control over drug dosing, because it scales with the amount of charge (i.e., the product of current and time) delivered to the skin.

Ultrasound is an oscillating pressure wave at a high frequency. The dominant effect is to disrupt stratum corneum lipid structure and thereby increase permeability. The effects of non-cavitational ultrasound on skin permeability enhance transfer of small, lipophilic compounds. Ultrasound can also generate cavitation, which is the formation, oscillation and, in some cases, collapse of bubbles in an ultrasonic pressure field. Cavitation is only generated under specific conditions, e.g., low-frequency ultrasound, that differ from those of ultrasonic heating or imaging devices. The opportunity for transdermal drug delivery is that cavitation bubbles concentrate the energy of ultrasound and thereby enable targeted effects at the site of bubble activity. Cavitation preferentially occurs within the coupling medium (e.g., a hydrogel) between the ultrasound transducer and skin.

Chemical enhancers, electroporation, cavitational ultrasound, microneedles, thermal ablation and microdermabrasion have been shown to deliver macromolecules, including therapeutic proteins and vaccines, across the skin. In particular, targeted enhancement of skin permeability in the stratum corneum is of interest. For example, dramatically increased enhancement with low skin irritation potential was found for a combination of sodium laureth sulfate and phenyl piperazine at concentrations of 0.35 and 0.15 wt %, respectively, in a 1:1 mixture of ethanol and phosphate-buffered saline. A natural pore-forming peptide, magainin, can be used to increase skin permeability by a mechanism proposed to target bilayer disruption in stratum corneum lipids and not in deeper tissue. The magainin is only effective when used in synergistic combination with a surfactant chemical enhancer, which served the dual purpose of increasing skin permeability to the drug as well as increasing penetration of magainin into the stratum corneum.

Electroporation has also been shown to disrupt lipid bilayer structures in the skin. Although the electric field applied for milliseconds during electroporation provides an electrophoretic driving force, diffusion through long-lived electropores can persist for up to hours, such that transdermal transport can be increased by orders of magnitude for small model drugs, peptides, vaccines and DNA. The electric field applied during electroporation is initially concentrated in the stratum corneum.

Thermal ablation selectively heats the skin surface to generate micron-scale perforations in the stratum corneum. Transiently heating the skin's surface to hundreds of degrees for microseconds to milliseconds localizes heat transfer to the skin surface without allowing heat to propagate to the viable tissues below. This spares these tissues from damage or pain. Mechanistically, thermal ablation may involve rapidly vaporizing water in the stratum corneum, such that the resulting volumetric expansion ablates micron-scale craters in the skin's surface. Skin heating has been achieved using ohmic microheaters and radio-frequency ablation. The microscopic length scales of localized skin disruption caused by thermal ablation have resulted in the procedure being well tolerated.

An additional way to remove the stratum corneum barrier employs abrasion by microdermabrasion or simply using sandpaper. Microdermabrasion is a widely used method to alter and remove skin tissue for cosmetic purposes. This abrasive mechanism, which is related to sand blasting on the microscopic scale, has been shown to increase skin permeability to drugs.

Topical formulations comprising the Rspo agonist can be administered to the region of skin comprising hair follicles, particularly skin of the scalp, but in some embodiments also comprising the skin of the eyelashes, eyebrows, beard, etc. The individual being treated may be male or female, usually a mammal, and usually a human. The individual may suffer from a hair loss condition, such as androgenic alopecia, or from alopecia resulting from various other causes. Alternatively, the individual can have normal hair growth who desires to have a promotion of hair growth or regulation of hair growth.

The compositions of the present invention can be for topical use and can be applied to the regions of skin comprising hair follicles, e.g. scalp, eyebrows, eyelashes, etc. The amounts and concentrations of the active agents in the compositions of the invention will vary depending on several different factors, including but not hereby limited to, the pH and condition of the skin; whether the skin is oily, dry, or in-between and the nature of the interaction between the various other agents to be included in the composition, but should be such to be effective while at the same time reducing the risk of untoward side effects, such as inflammation and unwanted change in the pigmentation of the hair or skin. Optimization of the concentration of the active agent(s), suitable for use with different skin types, which are used within the compositions of the invention, can be routinely determined by a skilled worker using well known methods that are commonly practiced within the art.

In general, the subject formulations may contain at least about 1 μg/ml active agent, at least about 10 μg/ml, at least about 50 μg/ml, at least about 100 μg/ml, at least about 500 μg/ml, and not more than about 50 mg/ml, usually not more than about 10 mg/ml. In some embodiments the formulation comprises at least about 0.1 mM, at least about 0.05, at least about 1 mM, at least about 5 mM, at least about 10 mM, at least about 50 mM, and not more than about 500 mM, not more than about 250 mM active agent. The active agents of the present invention are formulated at an effective concentration within the subject formulations, meaning at a concentration that provides the intended benefit when applied.

The Rspo agonist can be effective to promote anagen phase of the hair cycle, resulting in more rapid or prolonged hair growth. The Rspo agonist can be used for conditions in which regulating hair growth or promoting hair growth is desired. The compositions of the invention can be used for the topical treatment of a hair loss condition, such as alopecia in a mammal. The individual being treated may be a human, or may be an animal, e.g. canine, equine, bovine, etc. suffering from hair loss. In other embodiments the individual is a laboratory animal, e.g. rabbit, mouse, rat, etc., for purposes of evaluating treatments.

An Rspo agonist may be combined with any additional ingredient which may be active, functional, conventionally used in cosmetic, personal care or topical/transdermal pharmaceutical products or otherwise. A decision to include an additional ingredient and the choice of specific additional ingredients depends on the specific application and product formulation. Also, the line of demarcation between an “active” ingredient and an “inactive ingredient” is artificial and dependent on the specific application and product type. A substance that is an “active” ingredient in one application or product may be a “functional” ingredient in another, and vice versa.

In order to be effective in stimulating hair growth the composition may be formulated in such a way as to enhance the active agent's penetration of the skin. Accordingly, the composition may be formulated in conjunction with a skin penetration enhancing agent so as to better enable the active agent to deeply penetrate the epidermis of the skin, including particularly liposomal compositions. The formulations can include other components, such as buffering agents, lipophilic agents and cosmetically acceptable vehicles.

The formulations may be used in the form of gels, solutions, dispersions and emulsions, or may comprise carriers such as microneedles, macrocapsules, microcapsules, nanocapsules, macrospheres, microspheres, nanospheres, liposomes, oleosomes, chylomicrons, macroparticles, microparticles, nanoparticles, macrosponges, microsponges, nanosponges, powdered organic polymers, talcs, bentonites or other inorganic carriers.

The formulations may be used in any form employed in cosmetics or dermopharmacy: such as lotions, sprays, gels, hair styling products, hair holding products, sunscreens, sunblocks, emulsions, dispersions, solutions, milks, suspensions, scalp treatment lotions, or sprays.

### Skin Penetration Enhancing Agent

In addition to the liposomal component of the formulation, additional skin penetration agents may be included in the formulations of the invention. As used herein, a skin penetration enhancing agent is any factor that increases the penetration of the skin, preferably with minimal disruption to the acidic pH balance of the skin. Preferably, the skin penetration enhancing agent enhances the percutaneous delivery of the active agent into and through the layers of the skin, without providing substantial transdermal transmission of the active agent into the systemic circulation. The permeability enhancing agents of the invention are physio-chemically stable, do not have pharmacological effects, and have at least reduced irritancy or toxicity to the skin. When present in a composition of the invention, the amount of penetration enhancer is typically from about 1% to about 10% by weight of the total composition weight or from about 2% to about 5% by weight. The formulation and use of skin penetration enhancers in topical formulations is set forth generally in: PERCUTANEOUS PENETRATION ENHANCERS (Eric W. Smith & Howard I. Maibach eds. 1995); Ghosh, T. K. et al. 17 PHARM. TECH. 72 (1993); Ghosh, T. K. et al. 17 PHARM. TECH. 62 (1993); and Ghosh, T. K. et al. 17 PHARM. TECH. 68 (1993), all of which are hereby incorporated herein by reference in their entirety.

Suitable skin penetration enhancing agents include those agents that are capable of reducing the resistance of the skin to the active agent and promoting the active agent partitioning from the dosage form. Penetration enhancing agents may function in a variety of ways, including via the elution of the lipid and/or lipoprotein structures of the stratum corneum, by increasing lipid fluidity (e.g., by disrupting the tightly packed lipid chains), or by engaging in various protein interactions that result in a change in protein and/or lipid configuration that creates a passage for the active agent. Suitable topical skin permeability enhancing agents can be routinely selected for a particular use by those skilled in the art, and especially with reference to one of many standard texts in the art, such as Remington's Pharmaceutical Sciences, Vol. 18, Mack Publishing Co., Easton, Pa. (1990), in particular Chapter 87, which is hereby incorporated by reference in its entirety.

Accordingly, suitable skin penetration enhancing agents include but are not hereby limited to: sulfoxides, alcohols, polyols, fatty acids, esters, amides, surface active agents (such as pluronics, sulfates, lecithin, docusate sodium, polysorbates), water, and the like. Specifically, skin penetration enhancing agents include but are not hereby limited to dimethyl sulfoxide (DMSO), N-decylmethylsulfoxide, ethanol, phenyl ethanol, propylene glycol, lauric or myristic or palmitic or steric fatty acids, lauric acid, sodium laurate, neodecanoic acid, lauryl lactate, methyl laurate, hexamethylene lauramide, leucinic acid, oleic acid, capric acid, sodium oleate, sodium caprate, dodecyl-amine, cetryl lactate, myristyl lactate, isopropyl palmitate or isopropyl myristate esters, urea and derivatives, dodecyl N,N-dimethylamino acetate, hydroxyethyl lactamide, lecithin, phyophatidylcholine, sefsol-318 (a medium chain glyceride, surfactants, including polyoxyethylene (10) lauryl ether (Brij 361 R), diethyleneglycol lauryl ether (PEG-2-L), laurocapram (Azone; 1,1-dodecylazacycloheptan-2-one), acetonitrile, 1-decanol, 2-pyrrolidone, N-methylpyrrolidone, N-ethyl-1-pyrrolidone, 1-methyl-2-pyrrolidone, 1-lauryl-2-pyrrolidone, sucrose monooleate, acetone, polyethylene glycol 100-400 MW, dimethylacetamide, dimethylforamide, dimethylisosorbide, sodium bicarbonate, mentane, menthone, menthol, terpinene, D-terpinene, dipentene, N-nonalol, limonene, and various C₇₋₁₆ -alkanes in amounts that are safe and effective. A vasodilator that can be used in the formulations of the present invention is niacinamide (a vitamin B₃ compound), which aids in the penetration and uptake of active ingredients. The niacinamide may be used at a concentration of at least about 0.25% to 0.5%, more usually at least about 1%, and not more than about 5%.

### Buffering Agents

The normal pH of the skin is between about 4 and about 6.5, though it varies in people of different skin types. The compositions of the invention, therefore, in certain embodiments, should be formulated in such a manner so as to reduce the effects that the actual application of the composition has on the pH barrier of the skin and/or should be formulated in a manner so as to increase the penetration of the active agent. Accordingly, in certain embodiments the typical pH ranges for the compositions of the invention include a pH of about 3 to about 8, of about 4 to about 7, and more typically about 4.5 to about 6.5 or about 5. The desired pH ranges of the compositions of the invention can be obtained in accordance with practices well known in the art, for instance, by the inclusion of various buffering agents, which should be included in an amount and concentration to optimize the flux of the active agent through the skin surface and into the dermal layer of skin, while minimizing any possibility of skin irritation due to a change in the pH of the skin.

A conventional buffering agent such as a mixture of citric acid and trisodium citrate, may be added to stabilize the desired pH. Other buffering agents include, but are not limited to, sodium phosphate, monosodium dihydrogen phosphate, and disodium monohydrogen phosphate.

Various lipophilic agents may also be included as cosmetic benefit agents of the present invention in amounts that are safe and effective. A lipophilic agent to be added to a composition of the invention may be, for instance, a water-insoluble (hydrophobic) organic material or mixture of materials that are miscible with an Rspo agonist and are suitable for administration (e.g., topical administration) and formulated to enhance the penetration of an active agent of the invention. A lipophilic component may be in a range about 15% to about 40% by weight of the total composition weight or about 20% by weight.

Suitable lipophilic components are well known in the art and include, but are not limited to, vegetable, nut, and seed oils, such as almond oil, castor oil, coconut oil, corn oil, cotton seed oil, jojoba oil, linseed oil, grape seed oil, rape seed oil, mustard oil, olive oil, palm and palm kernel oil, peanut oil, safflower oil, sesame oil, soybean oil, sunflower-seed oil, crambe oil, wheat germ oil, and cocoa butter; animal oils and fats, such as lanolin, tallow, lard, beef fat, butterfat, mink oil, and fish oils; hydrocarbon and petroleum oils, such as petrolatum, mineral oil, and liquid paraffin. Additional lipophilic components include higher fatty acids such as lauric acid, myristic acid, palmitic acid, stearic acid, behenic acid, oleic acid, 12-hydroxystearic acid, undecylenic acid, tall acid, lanolin fatty acid, isostearic acid, linoleic acid and linolenic acid.

The lipophilic component may also include a suitable stiffening agent such as isopropyl myristate, glycerol monolaurate, glycerol monooleate, glycerol monolinoleate, isopropyl isostearate, isopropyl linoleate, isopropyl myristate/fatty acid monoglyceride combination, isopropyl myristate/ethanol/L-lactic acid combination, isopropyl palmitate, methyl acetate, methyl caprate or methyl laurate.

The formulation may also contain irritation-mitigating additives to minimize or eliminate the possibility of skin irritation or skin damage that may result from the penetration of the active agent or other components of the formulation. Suitable irritation-mitigating additives include, for example: a-tocopherol; monoamine oxidase blockers, particularly phenyl alcohols, such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the formulation at a concentration effective to mitigate irritation or skin damage, typically representing not more than about 20 wt %, more typically not more than about 5 wt %, of the formulation.

### Cosmetically Acceptable Vehicle

The formulation can comprise a cosmetically acceptable vehicle to act as a dilutant, dispersant or carrier for an active agent of the invention, so as to facilitate its distribution and uptake when the composition is applied to the skin and/or hair or scalp. Vehicles other than or in addition to water can include liquid or solid emollients, solvents, humectants, thickeners and powders.

The cosmetically acceptable vehicle will usually form from about 5% to about 99.9%, preferably from about 25% to about 80% by weight of the composition, and can, in the absence of other cosmetic adjuncts, form the balance of the composition.

The compositions may be in the form of aqueous, aqueous/alcoholic or oily solutions;

dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

A topical cosmetic composition of the invention will typically be formulated as a solution or gel which is prepared to be applied to the skin surface without friction, and which is typically a liquid or semiliquid preparation in which the active agent(s) of the invention are present in a lipid, alcohol or water base. Solutions are, typically, homogeneous mixtures prepared by dissolving one or more chemical substances (solute) in another liquid such that the molecules of the dissolved substance are dispersed among those of the solvent. The solution may contain other cosmetically acceptable chemicals to buffer, stabilize or preserve the solute. Commonly used examples of solvents used in preparing solutions are ethanol, water, propylene glycol or any other cosmetically acceptable vehicle, as for example, set forth below.

When the compositions of the invention are formulated as an emulsion, the proportion of the fatty phase may range from about 5% to about 80% by weight, and preferably from about 5% to about 50% by weight, relative to the total weight of the composition. Oils, emulsifiers and co-emulsifiers incorporated in the composition in emulsion form are selected from among those used conventionally in the cosmetic or dermatological field. The emulsifer and coemulsifier may be present in the composition at a proportion ranging from about 0.3% to about 30% by weight, and preferably from about 0.5% to about 20% by weight, relative to the total weight of the composition.

The compositions of the invention may also contain additives and adjuvants which are conventional in the cosmetic, pharmaceutical or dermatological field, such as gelling agents, active agents, preservatives, antioxidants, solvents, fragrances, fillers, bactericides, odor absorbers and dyestuffs or colorants. The amounts of these various additives and adjuvants are those conventionally used in the field, and, for example, range from about 0.01% to about 10% of the total weight of the composition. Depending on their nature, these additives and adjuvants may be introduced into the fatty phase or into the aqueous phase.

Another category of functional ingredients within the cosmetic compositions of the present invention are thickeners. A thickener will usually be present in amounts anywhere from about 0.1 to about 20% by weight, preferably from about 0.5% to about 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of about 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct components may also be incorporated into the cosmetic compositions. These ingredients may include coloring agents, opacifiers and perfumes. Specifically, these ingredients may include cosmetically suitable additives such as deionized water, hydrolyzed glycosaminoglycan, sodium hyaluraonate, triethanolamine, propylene glycol, methylparaben, propylparaben, acrylates, C10-C20 alkyl acrylate crosspolymers, C12-C15 alkyl benzoate, panthenol, biotin, sodium chloride, sodium phosphate and the like. Amounts of these other adjunct components may range anywhere from about 0.001% up to about 20% by weight of the composition.

The pharmaceutical compositions of the present invention may comprise a pharmaceutically acceptable carrier. Many pharmaceutically acceptable carriers may be employed in the compositions of the present invention. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

### Product Use, Form, and Packaging

In use, a quantity of the composition, for example from about 0.0001 ml to about 100 ml, from about 0.001 ml to about 10 ml, from about 0.01 ml to about 1 ml, typically about 0.1 ml is applied to a site of interest (i.e., skin or hair of the eyelash, eyebrow, and/or scalp) from a suitable container or applicator and, if necessary, it is then spread over the site. The product may be specifically formulated for use as a treatment for a specific area, e.g. the eyelashes, eyebrows, the face, the hair, or the scalp.

The cosmetic composition of the invention can be formulated in any form suitable for application to the site of interest). The composition can be packaged in a suitable container to suit its viscosity and intended use by the consumer. For example, a gel can be packaged in a bottle or a container fitted with a fine brush suitable controlled application to the lash line or eyebrow. The invention accordingly also provides a closed container containing a cosmetically acceptable composition as herein defined and may include a suitable applicator.

### Therapeutic Conditions

As described herein, a dose of an Rspo agonist is effective to promote anagen phase of the hair cycle, resulting in more rapid hair growth. An Rspo agonist can be used for conditions in which regulating hair growth or promoting hair growth is desired. Promoting hair growth can be performed on a subject with normal hair growth or a hair loss condition. Under normal hair growth conditions on the scalp, about 88% of the hairs are in the anagen phase, about 1% in catagen and the remainder in telogen. The compositions of the invention increase the percentage of hairs in anagen, thereby prolonging the growth phase of hair.

A hair loss condition is alopecia. “Alopecia,” as used herein is defined as loss of hair, includes, for example, alopecia areata, androgenic alopecia, etc. Hair loss is often a cause of great concern to the patient for cosmetic and psychological reasons. Hair grows in cycles. Each cycle consists of a long growing phase (anagen), a brief transitional apoptotic phase (catagen), and a short resting phase (telogen). At the end of the resting phase, the hair falls out (exogen) and a new hair starts growing in the follicle, beginning the cycle again. Normally, about 100 scalp hairs reach the end of resting phase each day and fall out. When significantly more than 100 hairs/day go into resting phase, clinical hair loss (telogen effluvium) may occur. A disruption of the growing phase causing abnormal loss of anagen hairs is an anagen effluvium. Besides the loss of hair, the length and diameter of each hair will be reduced in the adjacent areas even though the follicles remain intact.

Conditions of hair loss include alopecia areata, traction alopecia, trichotillomania, tinea capitis (fungal infection), telogen effluvium, and androgenic alopecia (“male-pattern baldness”, “female-pattern baldness”). Causes of alopecia include administration of chemotherapeutic agents and radiation, which impair or disrupt the anagen cycle. Other conditions resulting in hair loss include infection, systemic illnesses (particularly those that cause high fever, systemic lupus, endocrine disorders, and nutritional deficiencies). The compositions of the present invention find use in alleviating alopecia associated with these conditions.

Telogen effluvium is a transient, reversible, diffuse shedding of hair in which a high percentage of hair follicles enter the telogen phase prematurely as a result of physical or mental illness. Among the most important factors incriminated are childbirth, high fever, hemorrhage, sudden starvation, accidental or surgical trauma, severe emotional stress, and certain drugs.

Alopecia areata is an immunologic alopecia characterized by the abrupt onset of sharply defined areas of hair loss. In the most severe cases, the scalp will develop total hair loss (alopecia totalis) or the hair loss will involve the whole body surface (alopecia universalis). Most of the patients will run an unpredictable and relapsing course with multiple episodes of hair loss and regrowth. About 20 to 30 percent will have a single reversible episode. Regrowth of hair is common within several months, but in many instances is not complete, and relapses are common. Alopecia areata may be associated with autoimmune diseases such as vitiligo, pernicious anemia, collagen disease, and endocrinopathies.

Traumatic alopecia is induced by physical trauma, of which the two most important groups, from the therapeutic standpoint are trichotillomania and alopecia resulting from cosmetic procedures or improper hair care. Trichotillomania is a compulsive habit in which the individual repeatedly pulls or breaks off his or her own hair in a partially conscious state similar to thumb sucking or nail biting. Traumatic alopecia from cosmetic procedures is done consciously in ill-advised individuals and is almost exclusively seen among females. Sometimes this type of alopecia is associated with folliculitis induced by the occlusive effect of the oily cosmetics used in the procedure.

Anagen effluvium is a temporary alopecia caused by the inhibition of mitosis in the hair papilla by certain cytotoxic drugs, leading to constriction of the hair shaft or to complete failure of hair formation. In particular, alopecia frequently occurs in cancer patients who are treated with chemotherapeutic drugs and/or irradiation. Such agents damage hair follicles which contain mitotically active hair-producing cells. Such damage may cause abnormally slow growth of the hair or may lead to hair loss. While various attempts have been made to protect against alopecia or abnormal rates of hair growth during such treatments, there remains a need for an agent that prevents damage to hair follicles in a safe and effective manner.

Alopecia may also result from nutritional deficiencies and metabolic defects. Caloric deprivation must be very severe to produce hair loss. Increased shedding sometimes occurs after marked weight loss for obesity. Anemia, diabetes, hyper- and hypovitaminosis, and zinc deficiency may also lead to alopecia.

In some instances, a treatment regimen described herein increases the number of hair follicles by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases the number of hair follicles by 5% or more, by 10% or more, by 15% or more, by 20% or more, by 25% or more, by 30% or more, by 40% or more, by 50% or more, by 75% or more, or by 100% or more.

In some instances, a treatment regimen described herein increases the number of activated or stimulated hair follicles (e.g., NL, PEL or PELA follicular structures) by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases the number of activated or stimulated hair follicles (e.g., NL, PEL or PELA follicular structures) by 5% or more, by 10% or more, by 15% or more, by 20% or more, by 25% or more, by 30% or more, by 40% or more, by 50% or more, by 75% or more, or by 100% or more.

In some instances, the increase in number of hair follicles, activated or stimulated hair follicles, and/or NL, PEL, or PELA structures is observed in the treated area, for example, in an area of skin that was treated with Rspo agonist. In other embodiments, the increase in number of hair follicles, activated or stimulated hair follicles, and/or NL, PEL, or PELA structures is observed adjacent to the treated area. In other embodiments, the increase in number of hair follicles, activated or stimulated hair follicles, and/or NL, PEL, or PELA structures is observed in and adjacent to the treated area.

In some instances, measurement of hair follicles in accordance with the foregoing is within 3 days, or 4 days, 5 days, 6 days, 7 days, 8 days, 9 days, 10 days, 11 days, 12 days, 13 days, 14 days, 15 days, 16 days, 17 days, 18 days, 3 weeks, 4 weeks, or 1 month or longer after initiation of the treatment regimen. In one embodiment, measurement of hair follicles in accordance with the foregoing is based on a skin biopsy taken within 3 days, or 4 days, 5 days, 6 days, 7 days, 8 days, 9 days, 10 days, 11 days, 12 days, 13 days, 14 days, 15 days, 16 days, 17 days, 18 days, 3 weeks, 4 weeks, or 1 month or longer after initiation of the treatment regimen. In a particular embodiment, measurement of hair follicles in accordance with the foregoing is 11 days, 12 days, 13 days, 14 days, or 15 days, after initiation of the treatment regimen. In a particular embodiment, measurement of hair follicles in accordance with the foregoing is based on a skin biopsy taken 11 days, 12 days, 13 days, 14 days, or 15 days, after initiation of the treatment regimen.

In some instances, measurement of hair follicles in accordance with the foregoing provides a means for evaluating success of a method of Rspo agonist treatment (optionally in combination with other treatments). In an exemplary, non-limiting embodiment, success of a method of Rspo agonist treatment is determined based on a measured increase in total hair follicles in an area of skin subjected to Rspo agonist treatment, for example, compared to an area of skin that was not subjected to the Rspo agonist treatment. In another embodiment, success of a method of Rspo agonist treatment is determined based on a measured increase in activated or stimulated hair follicles, such as NL, PEL, or PELA follicular structures, in an area of skin subjected to Rspo agonist treatment, for example, compared to an area of skin that was not subjected to the Rspo agonist treatment step. In one embodiment, where a desired increase in hair follicles (or activated or stimulated hair follicles) is not observed, the treatment is discontinued. In another embodiment, where a desired increase in hair follicles (or activated or stimulated hair follicles) is not observed, Rspo agonist treatment is repeated. In another embodiment, where a desired increase in hair follicles (or activated or stimulated hair follicles) is not observed, Rspo agonist treatment is repeated using a different method (for example, switching from standard injections a transdermal patch or vice versa. In one embodiment, where a desired increase in hair follicles (or activated or stimulated hair follicles) is not observed, Rspo agonist treatment is repeated but with a higher dose, for example, increasing the dose by a factor of 2, 3, 3, 4, 5, 6, 7, 8, 9, or 10.

In some instances, measurement of hair follicles in accordance with the foregoing provides a means for evaluating whether a subject is a candidate for treatment, or continued treatment, with the methods described herein. In an exemplary, non-limiting embodiment, candidacy is established based on a measured increase in total hair follicles in an area of skin subjected to Rspo agonist treatment, for example, compared to an area of skin that was not subjected to the Rspo agonist treatment. In another embodiment, candidacy is established based on a measured increase in activated hair follicles, such as NL, PEL, or PELA follicular structures, in an area of skin subjected to Rspo agonist treatment, for example, compared to an area of skin that was not subjected to the Rspo agonist treatment. In one embodiment, where a desired increase in hair follicles (or activated hair follicles) is not observed, treatment of that particular subject is discontinued. In another embodiment, where a desired increase in hair follicles (or activated hair follicles) is not observed, Rspo agonist treatment is repeated. In another embodiment, where a desired increase in hair follicles (or activated hair follicles) is not observed, Rspo agonist treatment is repeated using a different dose. In one instance, a method of treatment is carried out over a small area of skin (e.g., 1×1 cm, or 1.5×1.5 cm, or 2×2 cm, or 2.5×2.5 cm, or 3×3 cm or more), hair follicles are measured in accordance with these methods, and if candidacy is established, the method of treatment is carried out over a larger area of skin, such as, e.g., an entire balding area of scalp.

In some embodiments, a treatment regimen described herein increases the anagen-to-telogen ratio by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases the anagen-to-telogen ratio by 5% or more, by 10% or more, by 15% or more, by 20% or more, by 25% or more, by 30% or more, by 40% or more, by 50% or more, by 75% or more, or by 100% or more. Such an increase in the anagen-to-telogen ratio may be measured within or after 1 month, 2 months, 3 months, 4 months, 5 months, 6 months, 7 months, 8 months, 9 months, 10 months, 11 months, or one year or longer after initiation of the treatment regimen.

In some instances, success of treatment is assessed by measuring hair count in a treated area of skin. For example, detectable hairs can be quantified by photography, e.g., by global photographic recording or phototrichographic analysis (as described in, e.g., Uno et al., 2002, Acta Venereol 82:7-12, incorporated herein by reference). Further, changes in the hair shaft thickness of photographically detectable hairs can be determined. In certain embodiments, the permanence of the hair growth is monitored over a time period of at least 2 months, 3 months, 4 months, 5 months, 6 months, 7 months, 8 months, 9 months, 10 months, 11 months, 1 year, 1.5 years, 2 years, 2.5 years, 3 years, 4 years, or at least 5 years or more.

In some instances, a treatment regimen described herein increases hair count by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases vellus hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases terminal hair by 5% or more, by 10% or more, by 15% or more, by 20% or more, by 25% or more, by 30% or more, by 40% or more, by 50% or more, by 75% or more, or by 100% or more. In some embodiments, a treatment regimen described herein results in 1-5%, 5-10%, 10-15%, 15-20%, 20-25%, 25-30%, 30-35%, 35-40%, 40-45%, 45-50%, 50-75%, or 75-100% conversion of vellus hair to nonvellus (i.e., intermediary or terminal hair). In some embodiments, a treatment regimen described herein increases hair thickness by 5% or more, by 10% or more, by 15% or more, by 20% or more, by 25% or more, by 30% or more, by 40% or more, by 50% or more, by 75% or more, or by 100% or more. In some embodiments, a treatment regimen described herein increases hair shaft diameter by approximately 1, 2, 2.5, 3, 3.5, 4, 4.5, 5, 5.5, 6, 6.5, 7, 7.5, 8, 8.5, 9, 10, 15, 20, 25, or 30 microns or more. In some embodiments, a treatment regimen described herein increases mean hair shaft diameter by 5% or more, by 10% or more, by 15% or more, by 20% or more, by 25% or more, by 30% or more, by 40% or more, by 50% or more, by 75% or more, or by 100% or more. In some embodiments, a treatment regimen described herein results in 1-5%, 5-10%, 10-15%, 15-20%, 20-25%, 25-30%, 30-35%, 35-40%, 40-45%, 45-50%, 50-75%, or 75-100% increase in mean hair shaft diameter. In some embodiments, a treatment regimen described herein increases the ratio of terminal to vellus hair follicles by 5% or more, by 10% or more, by 15% or more, by 20% or more, by 25% or more, by 30% or more, by 40% a or more, by 50% or more, by 75% or more, or by 100% or more. Such an improvement in hair count, vellus hair, terminal hair, conversion of vellus hair to nonvellus (e.g., intermediate or terminal) hair, hair thickness, hair shaft diameter, or the ratio of terminal to vellus hair may be measured within or after 1 month, 2 months, 3 months, 4 months, 5 months, 6 months, 7 months, 8 months, 9 months, 10 months, 11 months, or one year or longer after initiation of the treatment regimen.

In some instances, an Rspo agonist treatment described herein increases hair count by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases vellus hair by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases terminal hair by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein results in 1-5%, 5-10%, 10-15%, 15-20%, 20-25%, 25-30%, 30-35%, 35-40%, 40-45%, 45-50%, 50-75%, or 75-100% conversion of vellus hair to nonvellus (i.e., intermediary or terminal hair). In some embodiments, an Rspo agonist treatment described herein increases hair thickness by 5% or more, by 10% or more, by 15% or more, by 20% or more, by 25% or more, by 30% or more, by 40% or more, by 50% or more, by 75% or more, or by 100% or more. In some embodiments, an Rspo agonist treatment herein increases hair shaft diameter by approximately 1, 2, 2.5, 3, 3.5, 4, 4.5, 5, 5.5, 6, 6.5, 7, 7.5, 8, 8.5, 9, 10, 15, 20, 25, or 30 microns or more. In some embodiments, an Rspo agonist treatment described herein increases hair shaft diameter by 5% or more, by 10% or more, by 15% or more, by 20% or more, by 25% or more, by 30% or more, by 40% or more, by 50% or more, by 75% or more, or by 100% or more. In some embodiments, an Rspo agonist treatment described herein increases the ratio of terminal to vellus hair follicles by 5% or more, by 10% or more, by 15% or more, by 20% or more, by 25% or more, by 30% or more, by 40% or more, by 50% or more, by 75% or more, or by 100% or more. Such an improvement in hair count, vellus hair, terminal hair, conversion of vellus hair to nonvellus (e.g., intermediate or terminal) hair, hair thickness, hair shaft diameter, or the ratio of terminal to vellus hair may be measured within or after 1 month, 2 months, 3 months, 4 months, 5 months, 6 months, 7 months, 8 months, 9 months, 10 months, 11 months, or one year or longer after initiation of the treatment regimen.

In certain of the foregoing instances, the increase in hair count, vellus hair, terminal hair, conversion of vellus hair to nonvellus (e.g., intermediate or terminal) hair, hair thickness, hair shaft diameter, and/or the ratio of terminal to vellus hair is observed in the treated area, for example, in an area of skin that was treated with Rspo agonist. In other embodiments, the increase in hair count, vellus hair, terminal hair, conversion of vellus hair to nonvellus (e.g., intermediate or terminal) hair, hair thickness, hair shaft diameter, and/or the ratio of terminal to vellus hair is observed adjacent to the treated area. In other embodiments, the increase in hair count, vellus hair, terminal hair, conversion of vellus hair to nonvellus (e.g., intermediate or terminal) hair, hair thickness, hair shaft diameter, and/or the ratio of terminal to vellus hair is observed in and adjacent to the treated area.

### EXAMPLES

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### Example 1

### Targeting Hair Follicle Stem Cells via R-Spondin2 to Stimulate Hair Growth

Wnt signaling is involved in development of the hair follicle, and for inciting the growth (anagen) phase of the hair cycle. Using Axin2LacZ/+ reporter mice and RNA analyses, it was confirmed that Wnt signaling is elevated during the growth (anagen) phase of the hair cycle. Endogenous Wnt signaling was amplified in the skin and surrounding hair follicles via intradermal injection of R-Spondin2 (Rspo2) then demonstrated using Lgr5LacZ/+ mice that this enhanced Wnt environment activated Lgr5-positive stem cells in the hair follicle. The onset of catagen, which is marked by the cessation of cell proliferation and increased cell death, was repressed by Rspo2 injection, and the anagen phase persisted. As a consequence, hair grew longer. Rspo2 can act as a therapeutic protein to target hair follicle stem cells and promote hair growth.

Wnt-dependent activation of hair follicle stem cells was tested to determine if it was sufficient to stimulate hair growth. Because excessive cutaneous Wnt signaling is associated with neoplastic conditions including pilomatricomas and basal cell carcinomas, an approach was used that did not introduce Wnt protein itself into the skin but instead amplified the endogenous Wnt signal. R-spondin2 protein (RSPO2) is a secreted protein that can modulate Wnt signaling by binding to and stabilizing the Wnt receptors Lrp5/6 on the cell surface. Stabilization of Lrp5/6 in turn can prevent degradation of Wnt intracellular mediator beta catenin, which ultimately prolongs Wnt signalling.

Here, it was demonstrated that intradermal injection of Rspo2 is sufficient to prolong anagen and delay the onset of catagen. Rspo2 achieves this effect, at least in part, by up-regulating the expression of Wnt target genes Axin2 and Lgr5 and by repressing the expression of the Wnt antagonist Dkk1. Daily intradermal injections of Rspo2 inhibit cell death associated with catagen, and thus prevent hair follicles from entering catagen. The end result is significantly longer and thicker hair.

Endogenous Wnt Signaling Levels Fluctuate with the Hair Cycle.

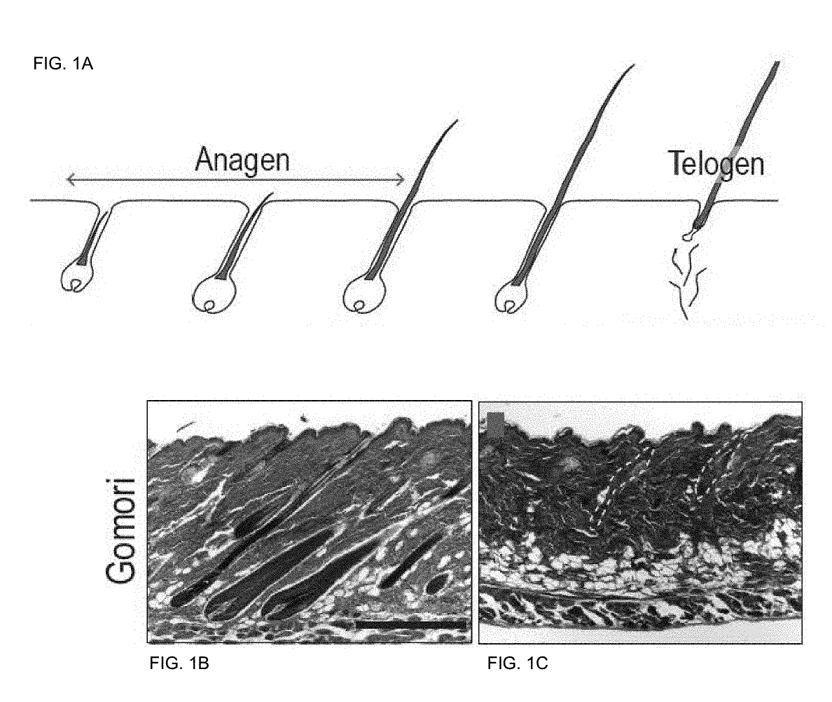

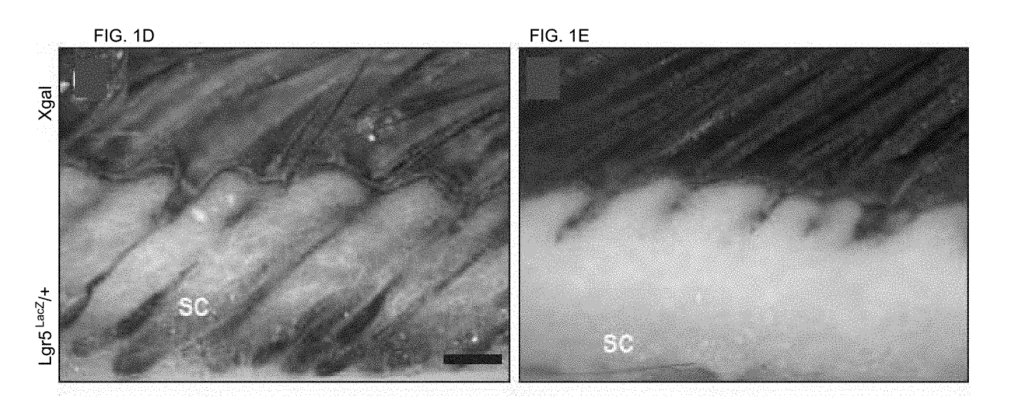

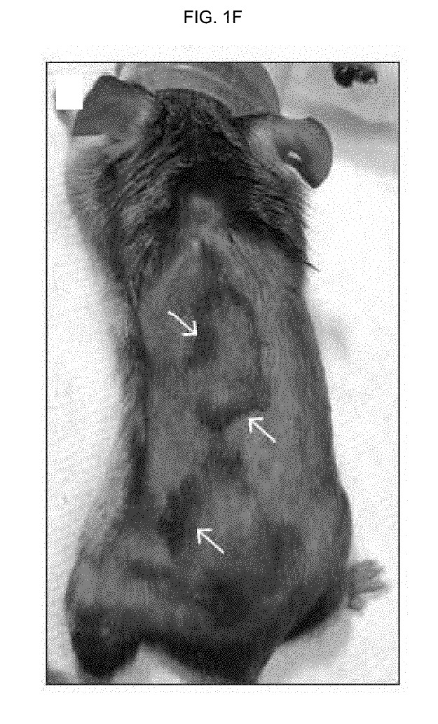

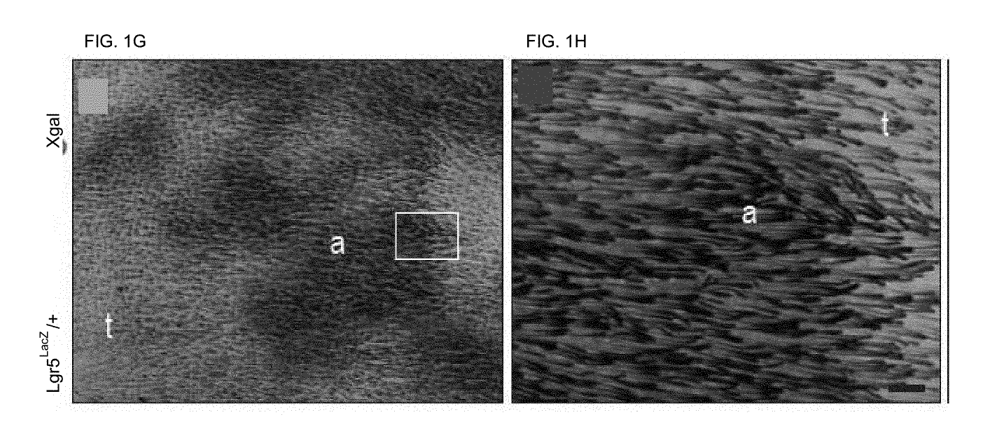

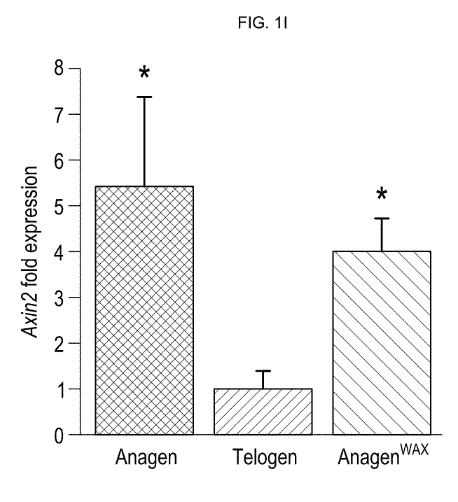

Anagen hair follicles, with their elongated morphology and their dermal papilla positioned in subcutaneous fat (FIG. 1A) are easily distinguished from miniaturized telogen hair follicles (FIG. 1B). Although a TOPgal Wnt reporter strain has been developed to track Wnt signaling in the skin and hair follicle, the strain suffers from two major problems: first, the reporter is minimally active in adult skin, and after skin wounding. Second, because the strain was generated by random insertion it shares risks common to most transgenic lines including inconsistent reporter activity from generation to generation. Therefore, Axin2^{LacZ/+} mice were used, which show robust Xgal staining in tissues known to be Wnt responsive. It was confirmed strong Xgal staining in anagen hair follicles (FIG. 10) and minimal Xgal staining in telogen hair follicles from Axin2^{LacZ/+} mice (FIG. 1D).

A second method was used to confirm that endogenous Wnt signaling was highest in anagen hair follicles. At 5 weeks of age the murine hair cycle is synchronized but as mice age the hair cycle becomes asynchronous. Shaving the dorsal surface of the mouse reveals this asynchronicity (FIG. 1E). Whole-mount Xgal staining of an Axin2^{LacZ/+} pelt revealed patches of intense Xgal staining intermixed with patches of weak coloration (FIG. 1F). The areas of intense Xgal staining corresponded to regions where the hair follicles were in anagen whereas the areas of weaker staining corresponded to telogen hair follicles (FIG. 1G). Using a third, quantitative method confirmed that endogenous Wnt signaling is highest in the skin of 5 week-old mice whose synchronized hair follicles are in anagen and lowest in the skin of 8 week-old mice whose hair follicles are in telogen (FIG. 1H). Finally, endogenous Wnt responsiveness as measured by expression of the Wnt target gene Axin2 was demonstrated to be highest in skin that has been waxed to induce the anagen phase of the hair cycle (FIG. 1H).

### Reduced Wnt Signaling Permits Catagen

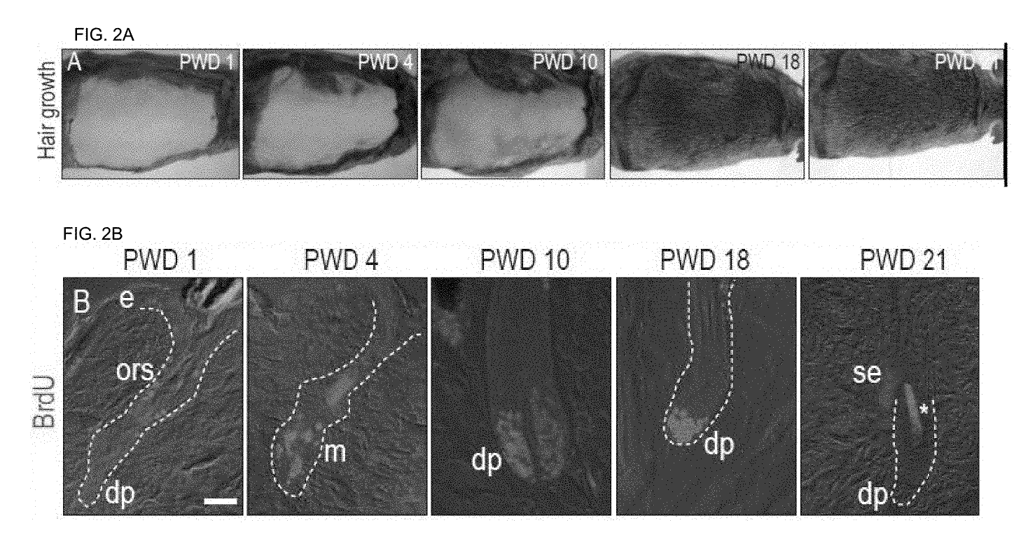

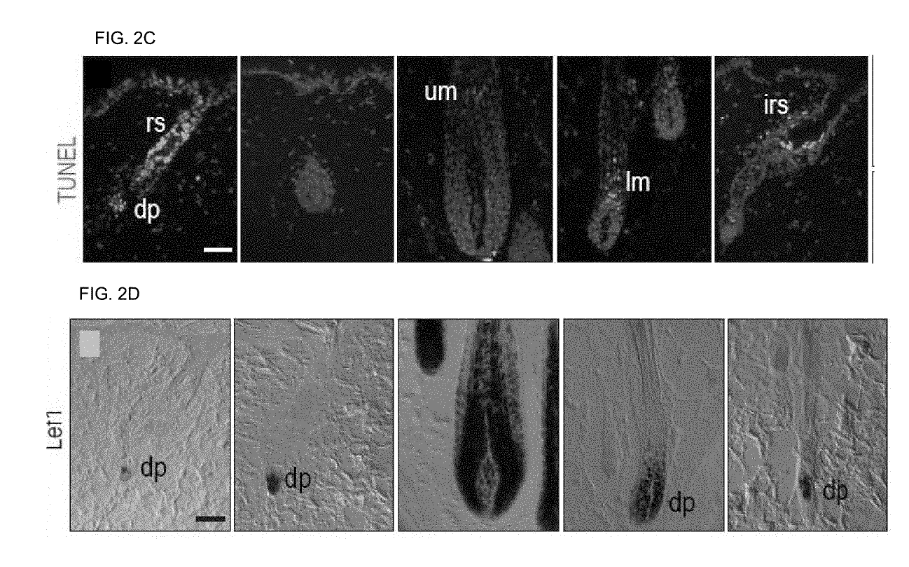

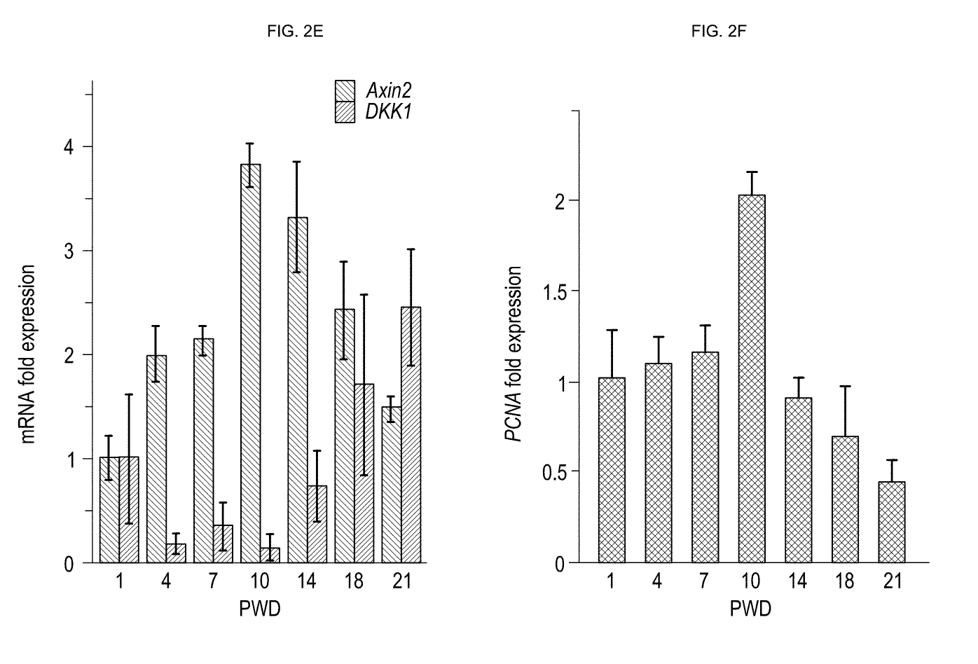

Rspo2 was used to amplify endogenous Wnt signaling and in doing so, repress and delay the onset of catagen. Consequently, the normal onset of catagen during synchronized hair growth was detected as follows. Age-matched, male CD1 mice were employed for this study; the dorsal skin was waxed (FIG. 2A) and profiled by histology from post-waxing day 1 (PWD1) until PWD21, at which time hair follicles were in telogen and the coat was fully regrown (FIG. 2A).

Catagen is distinguished by the lack of cell proliferation within the hair follicle. BrdU incorporation demonstrated a decline in cell proliferation on PWD18 (FIG. 2B). Before that time point (i.e., PWD1-14), abundant BrdU^{+ve} cells were present in matrix cells and the outer root sheath (FIG. 2B). Catagen is also distinguished by increased programmed cell death in the hair follicle. TUNEL staining was used to demarcate the onset of apoptosis: on PWD1, immediately after removal of the hair shaft, extensive TUNEL staining was observed (FIG. 2C). Very low levels of TUNEL staining were detected on PWD4; on PWD10, TUNEL^{+ve} cells are restricted to the upper matrix (FIG. 2C). From PWD18 onwards there was a sharp increase in TUNEL^{+ve} cells in the lower matrix and the inner root sheath (FIG. 2C).

We also profiled expression of the Wnt target protein, Lef1. Lef1 was tightly restricted to the dermal papilla on PWD1 (FIG. 2D) then gradually extended to the matrix cells on PWD7 until expression reached its vertex on PWD10 (FIG. 2D). Thereafter, the number of Lef1^{+ve} cells diminished, reaching its lowest level on PWD21 (FIG. 2D). Collectively, these data demonstrate that between PWD1 and 10, hair follicles were in a Wnt-high state of anagen and by PWD18, Wnt signaling declined and hair follicles transitioned to catagen.

We used a complementary, quantitative method to interrogate the state of Wnt responsiveness after waxing. Throughout the 21-day post-waxing period 4 mm punch biopsies were collected for qRT-PCR analyses. PCNA expression was used as an internal positive control, and its expression level paralleled the BrdU analysis, verifying that anagen extended from PWD4-10 and catagen onset occurred between PWD14-18 (FIG. 2E). Axin2 expression followed the same trend, remaining at or near baseline until PWD4, peaking on PWD10, and declining after PWD14 (FIG. 2F). Expression of the Wnt antagonist Dkk1 was lowest during anagen (PWD4-10) and gradually increased at the onset of catagen (PWD14). Collectively, these histology, immunohistochemical, and quantitative measurements verified the onset of anagen by PWD4, and the onset of catagen by PWD14.

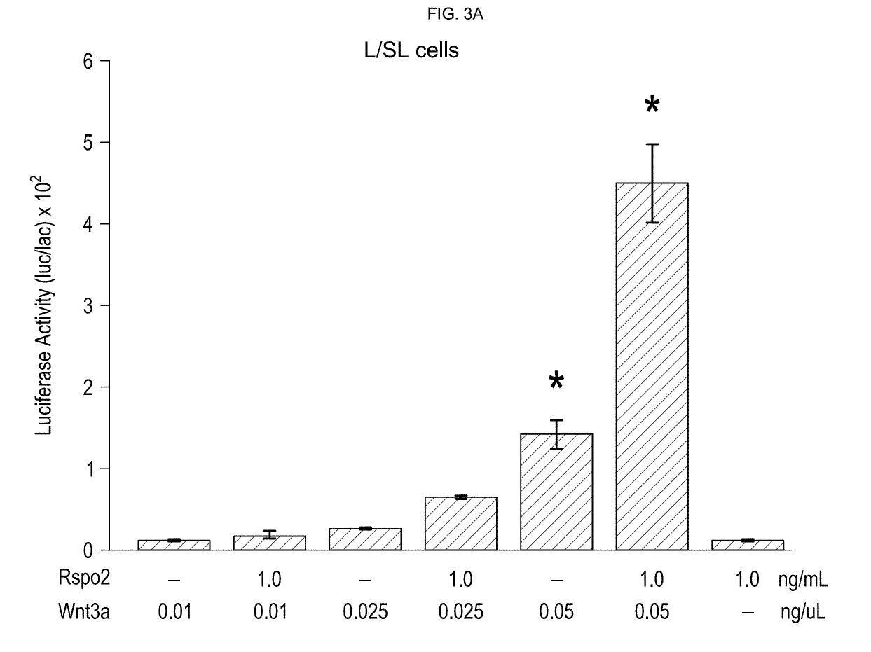

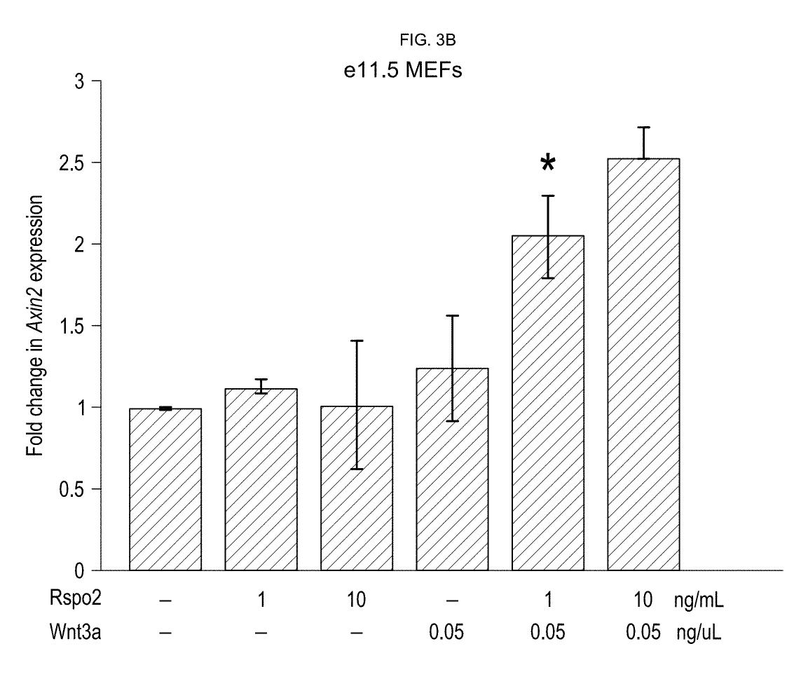

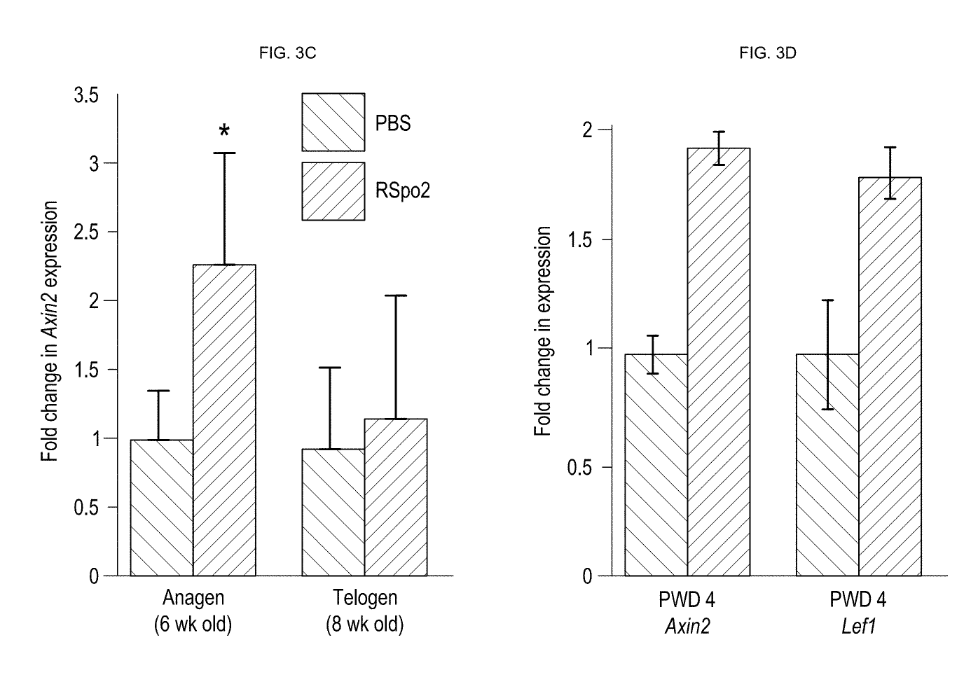

Rspo2 amplifies exogenous and endogenous Wnt signalling. Rspo2 amplifies Wnt signalling, a finding verified using the LSL reporter cell. The minimum Wnt concentration needed for pathway activation in LSL cells was determined, then augmented this minimal Wnt stimulus with a minimum concentration of Rspo2. For example, 0.025 ng/μL Wnt3a is insufficient to activate luciferase activity in LSL cells, but the addition of 1.0 ng/mL of Rspo2 amplifies luciferase activity by 2.3-fold (FIG. 3A). A 0.05 ng/μL dose of Wnt3a is amplified 5-fold by the addition of 1.0 ng/mL Rspo2 (FIG. 3A).

LSL cells, however, are engineered to be maximally sensitive to Wnt ligands and Wnt agonists and therefore provide little meaningful data on the relationship between dose and drug effect. We therefore turned to primary cultures of mouse embryonic fibroblasts (MEFs) to gain insights into the minimum Wnt stimulus required for Rspo2-mediated amplification of Wnt pathway activity in native cells. MEFs treated with 10 ng/mL Rspo2 alone did not show an increase in Axin2 expression above baseline (FIG. 3B), indicating the absence of endogenous Wnt ligands in the culture condition. The addition of 0.05 ng/μL Wnt3a to the MEF culture was also insufficient to increase Axin2 levels above baseline (FIG. 3B). The combination of 0.05 ng/μL WNT3A and 1 ng/mL Rspo2 however was sufficient to elevate Axin2 expression two-fold (FIG. 3B). Increasing Rspo2 to 10 ng/mL elevated Axin2 expression 2.5-fold (FIG. 3B).

The addition of 0.05 ng/μL exogenous WNT3A was required in order to observe an Rspo2-mediated increase in Wnt signaling. To determine if endogenous Wnt levels were sufficient to allow an Rspo2-mediated amplification of endogenous Wnt signaling. To test this possibility, Rspo2 protein (2.5 μL of a 100 ng/μL solution) was delivered via intradermal injection into the dorsum of 6 week-old mice, whose first hair cycle is synchronized and is in anagen. As a control the same concentration of Rspo2 was injected intradermally into 8 week-old mice whose hair is also synchronized but is in telogen. Punch biopsies were collected 24 h after Rspo2 injection and interrogated by qRT-PCR for Axin2 expression. These analyses revealed that during the Wnt-high state of anagen, Rspo2 injections resulted in 2-fold amplification in endogenous Wnt signaling (FIG. 3C). Rspo2 injections did not stimulate Axin2 expression above baseline during the Wnt-low state of telogen (FIG. 3C). Thus, when delivered in vivo Rspo2 did not activate endogenous Wnt pathway activity in the hair follicle unless there was an endogenous Wnt signal.

There may be some unique features of age-dependent anagen that are not shared adult hair follicles that are induced to enter anagen by waxing or plucking. Therefore it was directly tested whether Rspo2 injections were sufficient to amplify endogenous Wnt signaling after waxing. PWD4 was the earliest time point after waxing when endogenous Wnt signaling is elevated above baseline (FIG. 2) and Rspo2 injections at this timepoint were sufficient to induce a 2-fold increase in Axin2 and Left expression (FIG. 3D). Rspo2 injections at later time points did not result in an increase in Axin2 expression (not shown). Thus, when delivered to a tissue with active Wnt signaling, Rspo2 injections increase endogenous Wnt target gene expression 2-fold. Our next experiments were directed at determining whether this 2-fold amplification was sufficient to induce a phenotypic response in the hair follicle.

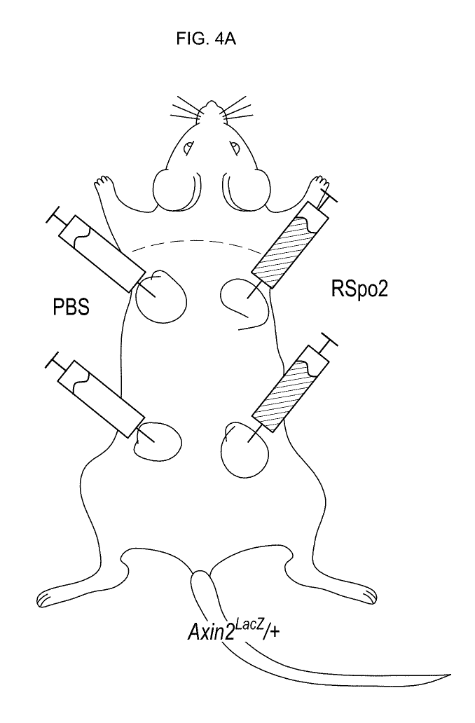

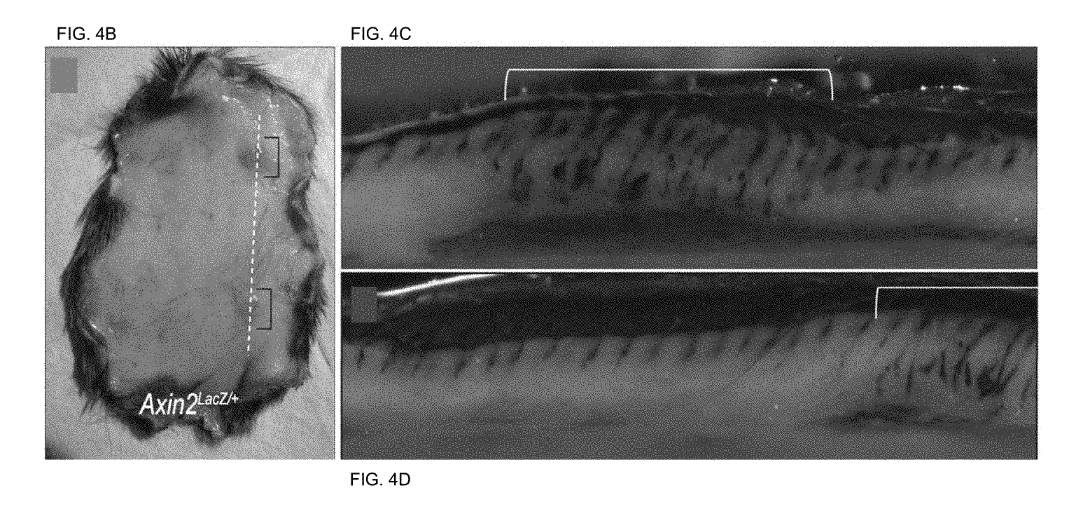

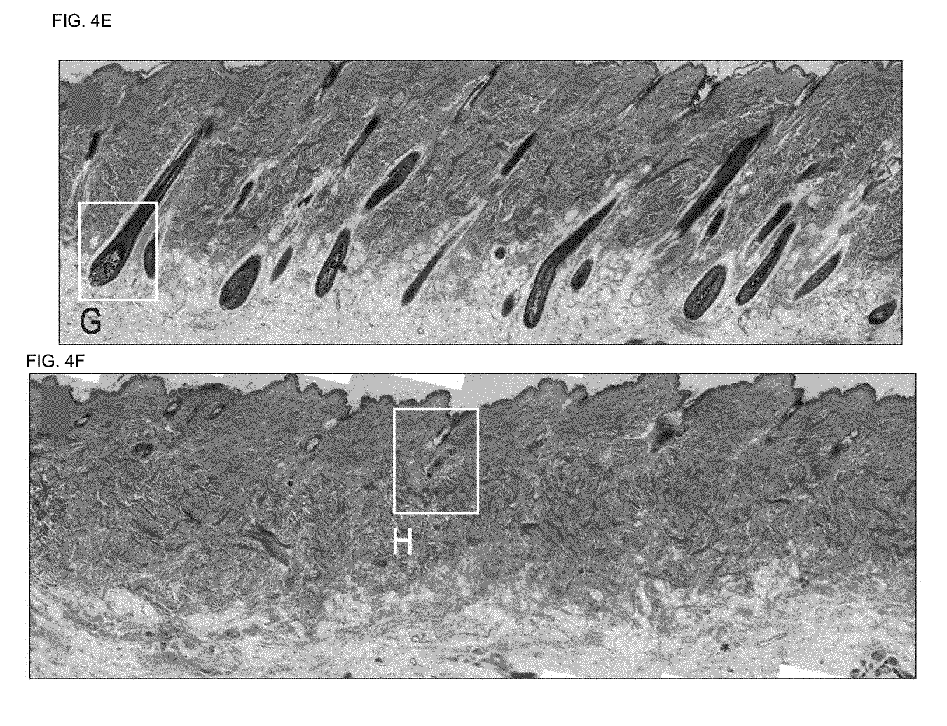

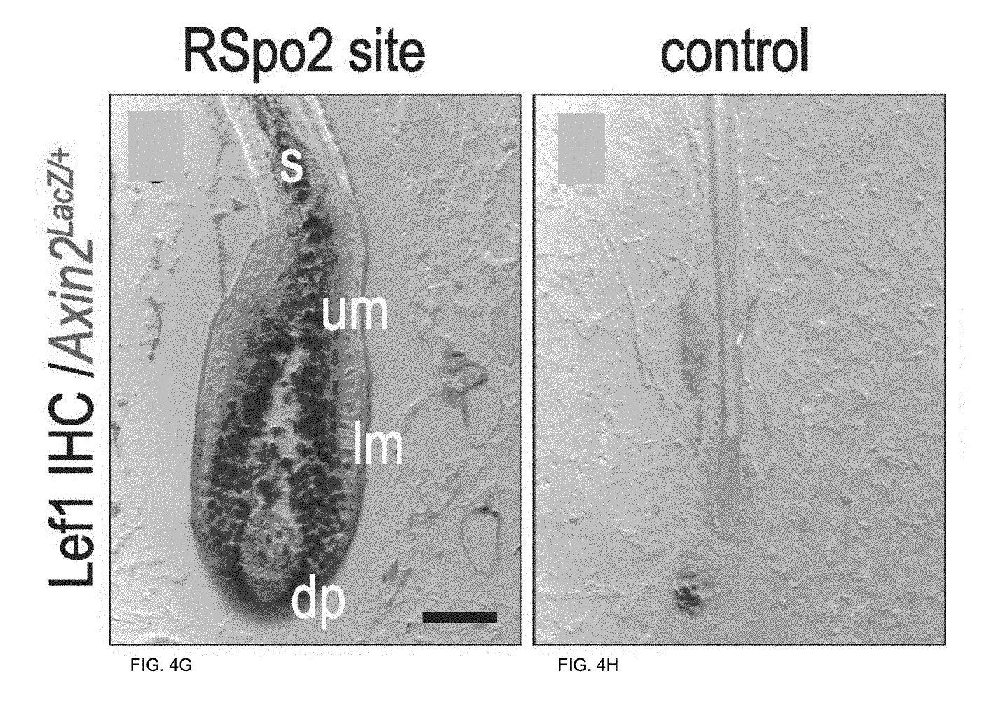

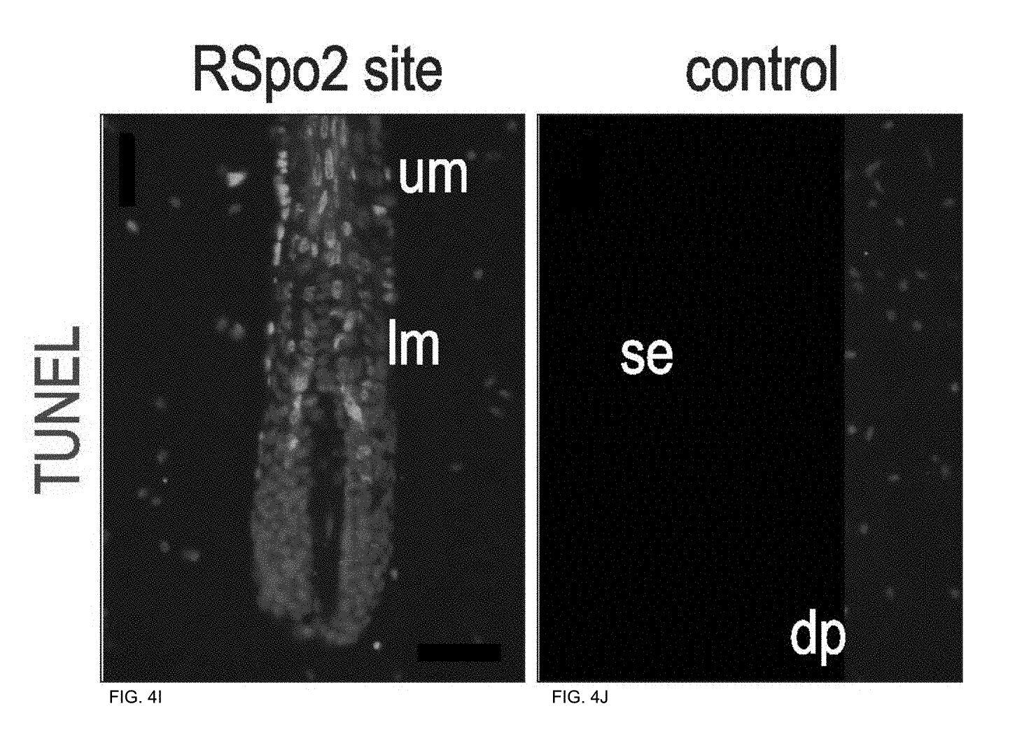

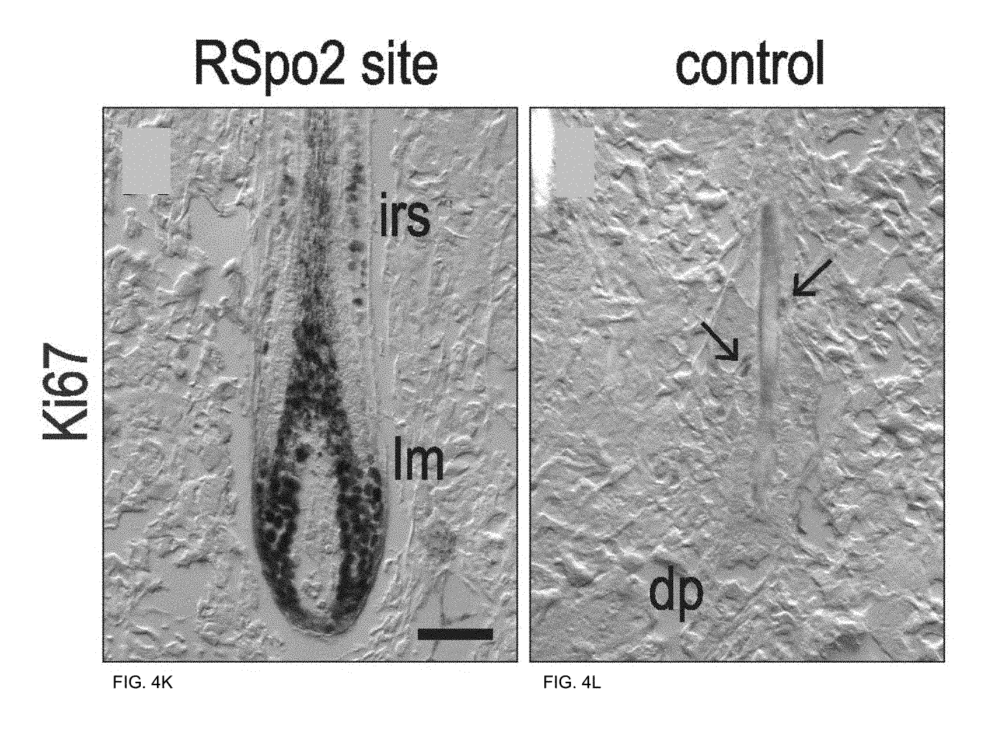

Rspo2 prolongs anagen. The backs of Axin2^{LacZ/+} mice were waxed and on PWD4, each Axin2^{LacZ/+} mouse received four intradermal injections: two PBS (control) injections (2.5 μL) on one side and two Rspo2 injections (2.5 μL of a 100 ng/μL solution) on the contralateral side (N=5 mice; FIG. 4A). Mice received daily injections from PWD4-10. At the onset of telogen (i.e., PWD22) pelts were harvested (FIG. 4B) and subjected to Xgal staining. From the subdermal surface two areas were immediately distinguishable, which corresponded to the sites of Rspo2 injection (FIG. 4B, brackets). Unlike the rest of the pelt, the hair follicles were strongly Xgal^{+ve} and were clearly in anagen (FIG. 4C). At all other sites in the pelt hair follicles were in catagen, as evidenced by their miniaturization and weak Xgal staining (FIG. 4D).

We examined various regions of the pelts from Axin2^{LacZ/+} mice using histology. At the Rspo2 injection sites, anagen hair follicles had an elongated morphology (FIG. 4E) whereas throughout the remainder of the pelt miniaturized hair follicles were in telogen (FIG. 4F).

At Rspo2 injection sites, Xgal^{+ve} hair follicles were immunopositive for Lef1 (FIG. 4G). In contrast, at PBS injection sites and elsewhere in the area that was waxed, hair follicles had progressed to the catagen phase and only the dermal papilla remained weakly positive for Lef1 immunostaining (arrow, FIG. 4H). Markers of programmed cell death and cell proliferation were used to confirm the phase of the hair follicles: at the Rspo2 injection sites, TUNEL staining was restricted to the upper and lower matrix of hair follicles (FIG. 4I). In contrast, hair follicles elsewhere in the pelt showed evidence of TUNEL staining both in cells of the dermal papilla and the sebaceous gland (FIG. 4J). At the Rspo2 injection sites Ki67 immunostaining was detected in proliferating cells of the matrix and the inner root sheath (FIG. 4K). Very few proliferating cells were detectable in telogen hair follicles throughout the remainder of the pelt (FIG. 4L). Thus, intradermal delivery of Rspo2 prevented the onset of catagen and kept hair follicles in anagen, long after the remaining synchronized hair follicles had progressed to telogen.

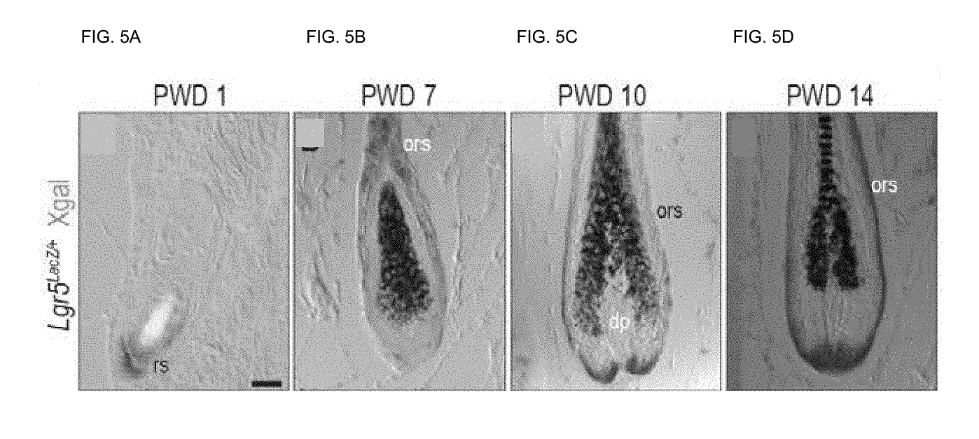

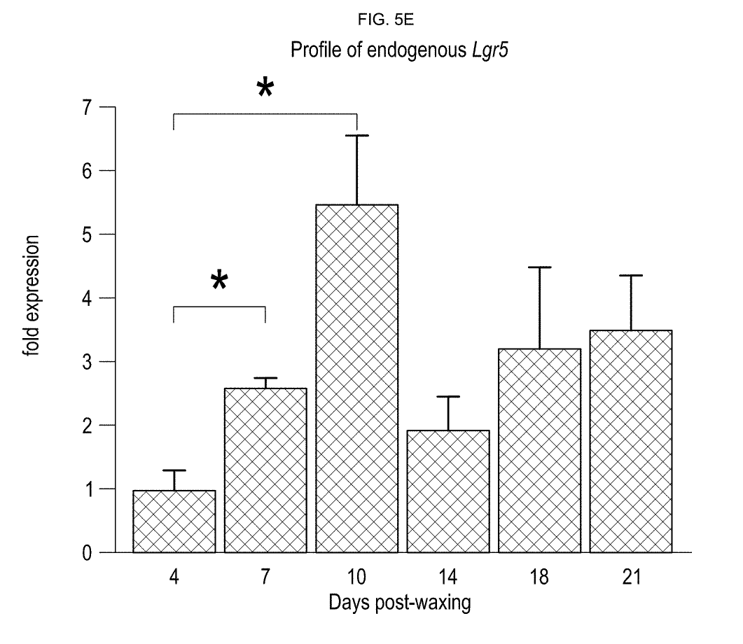

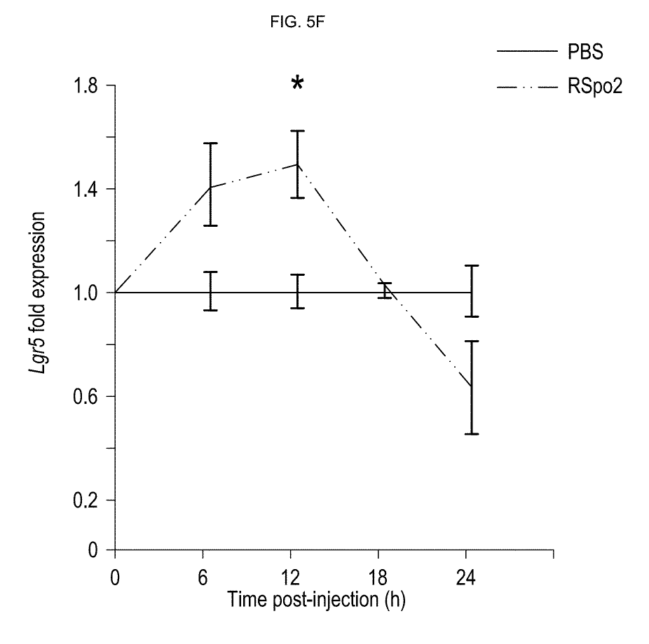

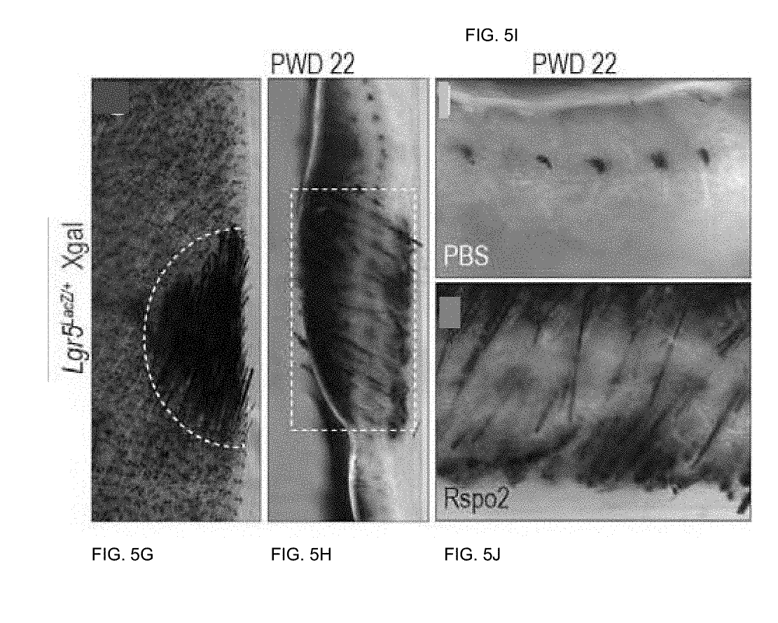

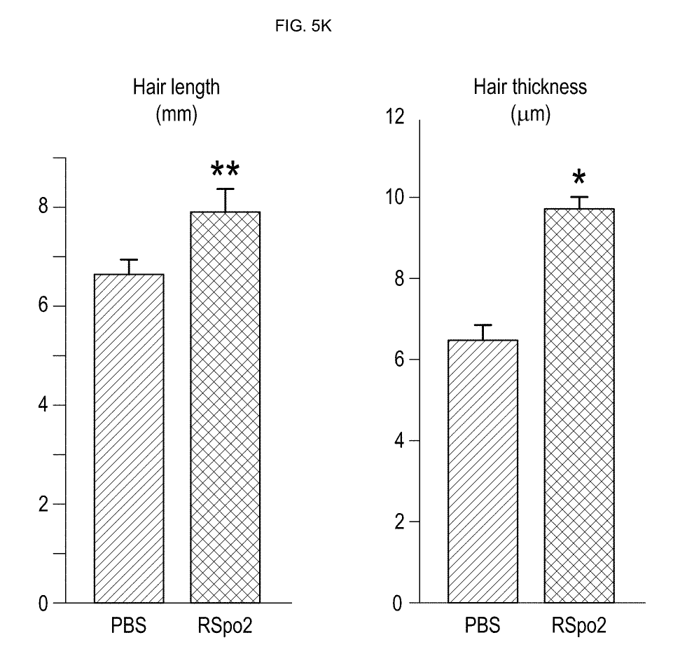

Rspo2 stimulates hair growth via activation of Lgr5 stem cells in the hair follicle Intradermal delivery of Rspo2 delayed the onset of catagen, thereby lengthening the growth phase of the hair cycle. Further experiments were performed to clarify the mechanism(s) by which Rspo2 achieved this effect. Hair follicle stem cells are typically maintained in a quiescent state during catagen and telogen by BMP signaling, and only proliferate early in anagen. Lgr5 marks these proliferative stem cells in the hair follicle and using Lgr5^{LacZ/+} mice the spatial and temporal distribution of Lgr5^{+ve} cells after waxing was confirmed. On PWD1, Lgr5^{+ve} cells were the outer root sheath and dermal papilla (FIG. 5A); on PWD7, Lgr5^{+ve} cells more abundant in the outer root sheath (FIG. 5B). On PWD10 through PWD14, Lgr5^{+ve} cells filled the outer root sheath (FIG. 5C,D respectively). Quantification of Lgr5 expression using qRT-PCR correlated with the observed increase in Lgr5^{+ve} cells; from PWD1, Lgr5 expression increased until PWD 10, after which it returned to baseline (FIG. 5E). This corresponds to the phases of anagen and catagen determined previously (FIG. 2).

Further experiments were performed to test whether Rspo2 regulated Lgr5 expression. At various time points after a subdermal Rspo2 injection tissues were interrogated by qRT-PCR. These analyses demonstrated that Lgr5 expression was transiently elevated in response to Rspo2 injection (FIG. 5F). To determine whether Rspo2-induced Lgr5 expression extended anagen. Rspo2 was injected intradermally into Lgr5^{LacZ/+} mice for 6 days beginning on PWD4; on PWD22, the pelt was bisected and examined by whole-mount Xgal staining. PBS-injected sites were indistinguishable from the rest of the pelt; Rspo2 injection sites were prominent (dotted line, FIG. 5G). Examination of the cut edge revealed that hair follicles in the Rspo2 injection site were Xgal^{+ve} and were in anagen, while surrounding hair follicles were in telogen (FIG. 5H). PBS-treated hair follicles and untreated regions of the Lgr5^{LacZ/+} pelt had miniaturized telogen stage hair follicles that were weakly positive for Xgal (FIG. 5I) whereas hair follicles in the Rspo2 injection sites remained in anagen and were robustly Xgal^{+ve} (FIG. 5J). When hair shafts were measured, those in the Rspo2 injection sites grew on average 1.2 mm longer than those in the remainder of the pelt, which represented an 18% increase in hair growth over a 22-day period (FIG. 5K). Rspo2 hair follicles were also thicker, by 54%.

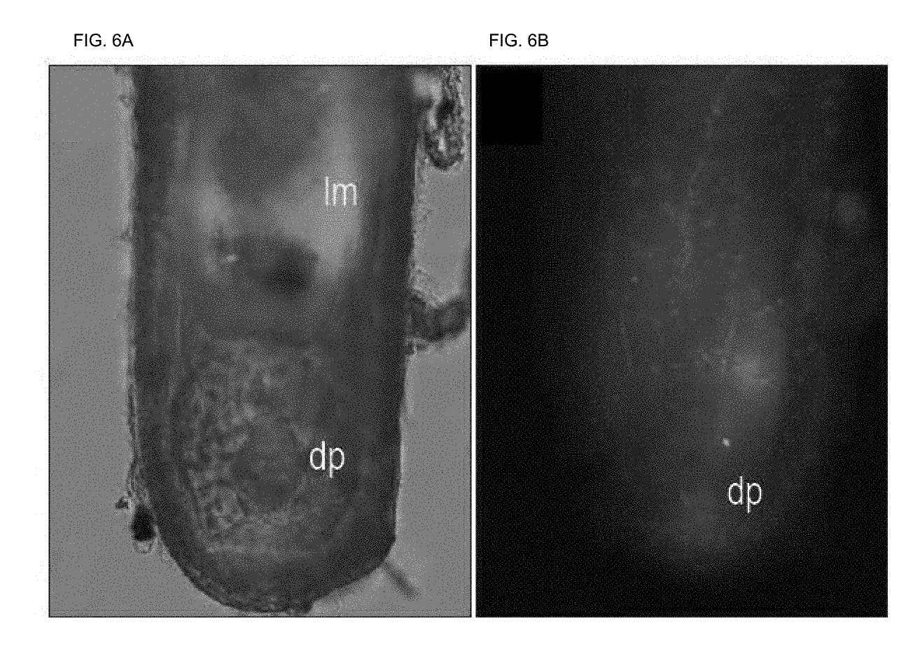

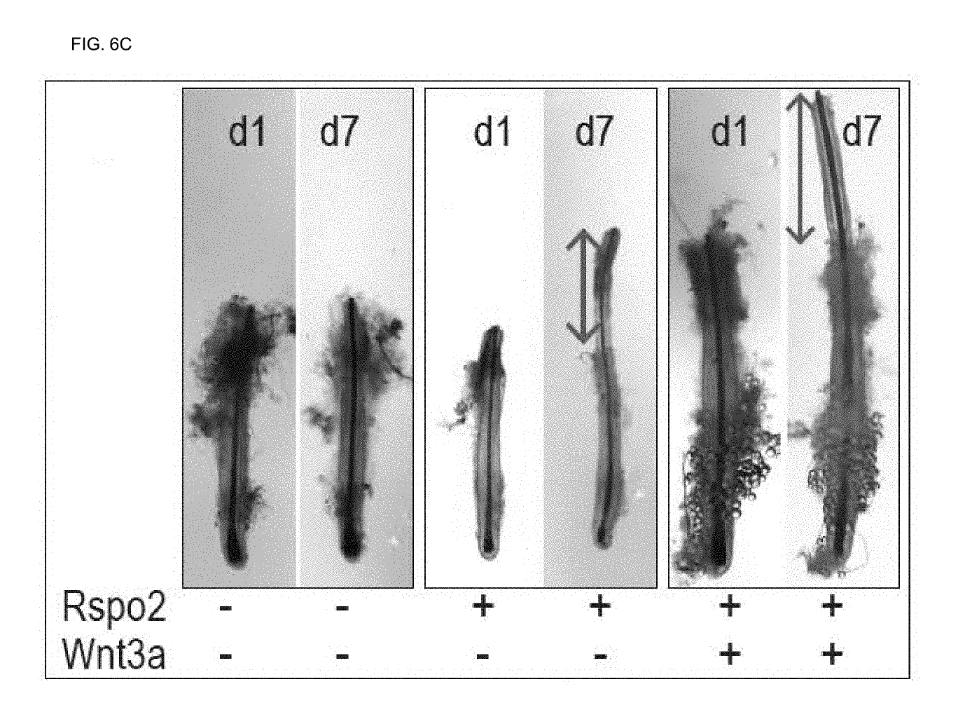

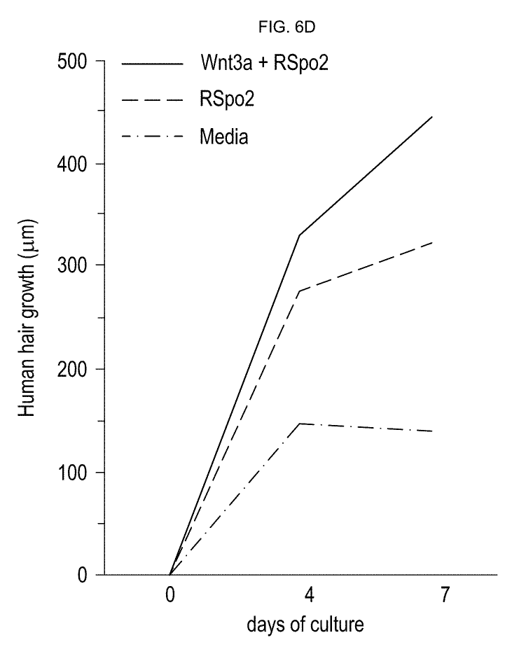

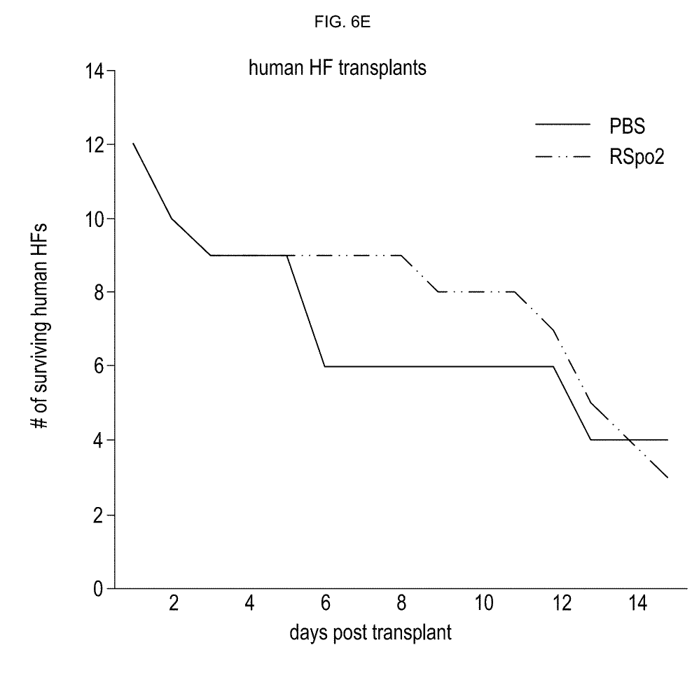

Human hair growth is enhanced by WNT3A +Rspo2. Given the dramatic increase in hair follicle length in response to Rspo2 injections, experiments were performed to test whether human hair follicles exhibited a similar response. Hair follicles were harvested from the scalps of male volunteers and immediately cultured in hair growth media, Rspo2 (2 ng/μL) or WNT3A (0.15 ng/μL)+Rspo2 (2 ng/μL). The growth of individual hair follicles was monitored over a 14-day time period (FIG. 6).

Human hair follicles cultured in the presence of Rspo2 alone did not show significantly more growth than those in media alone; however those grown in the presence of WNT3A+Rspo2 showed significantly more growth). These data strongly suggest that cultured human hair follicles do not produce sufficient amounts of WNT to enable amplification of their endogenous signal by Rspo2. Hair follicles maintained their morphology over the entire culture period (FIGS. 6B and 6C) but when their rate of growth (FIG. 6C-D) and the percent surviving hair follicles (FIG. 6E) were assessed, it was clear that the maximum effect of the WNT treatment was observed at earlier time points. Therefore, the overall growth rate of human hair follicles is not improved but their survival in vitro is transiently prolonged by Rspo2 treatment alone.

Current treatment options for hair loss are limited; topical products, such as minoxidil and finasteride, retard hair shedding (effluvium) but do not stimulate new hair growth. Hair transplants, where hair follicles are harvested from one part of the scalp and moved to another, are effective but upon harvesting of the hair follicles, a subset of them undergo apoptosis. Consequently, when those follicles are transplanted the hair shaft falls out.

Here, a R-spondin-based strategy was used to enhance endogenous Wnt signaling and in doing so, extend anagen. Intradermal R-spondin amplifies endogenous Wnt signaling 2-3 fold (FIGS. 2,3), which was sufficient to prolong anagen (FIGS. 4,5) via an increase in Lgr5 expression and Lgr5 positive cells(FIG. 5). Rspo2 treatment resulted in longer, thicker hair shafts (FIG. 5).

A number of groups have demonstrated that viral over-expression of Wnt ligands induces anagen. These groups also found that elevated Wnt signaling controls the size of the hair shaft. Wnt induced hair shafts are thicker but not longer, indicating that the onset of catagen remains unaffected by Wnt over-expression. So in order to get longer hair, one can prolong the anagen phase of hair growth and inhibit the onset of catagen.

Dkk1 is a regulator of catagen. For example, see Binnerts et al. (2007) Proc Natl Acad Sci U S A. 2007 Sep. 11; 104 (37):14700; and Kwack et al. (2008) J. Invest. Dermatol. 128(2):262-9, each herein specifically incorporated by reference.

### Materials & Methods

Animals. The Stanford Committee on Animal Research approved all procedures. Axin2^{LacZ/+} mice were obtained from Jackson Laboratory (Bar Harbor, Me.).

Rspo2 preparation and delivery. Lyophilized recombinant RSpo2 (R&D Systems) was re-suspended to 200 ng/μL with sterile PBS. This was diluted to 100 ng/μl for injection and diluted to 2 ng/μL in media for hair cultures. All injections were intradermal using a 17G insulin syringe.

Activity assay. Mouse LSL cells are stably transfected with a Wnt-responsive luciferase reporter plasmid pSuperTOPFlash (Addgene) and a constitutive LacZ expression construct pEF/Myc/His/LacZ (Invitrogen) for normalizing beta galactosidase activity to cell number. Cells (50000 cells/well, 96-well plate) were treated with PBS or Wnt3a in DMEM supplemented with 10% FBS (Gibco) and 1% P/S (Cellgro) at concentrations indicated. Cells were incubated 18 hr at 37° C., 5% CO₂, then washed, lysed with Lysis Buffer (Applied Biosystems), and the luciferase and β-galactosidase expression levels quantified using a dual-light combined reporter gene assay system (Applied Biosystems). Bioluminescence was quantified with triplicate reads on a dual-light ready luminometer (Berthold). Wnt signaling activity is defined from a standard curve generated by serial dilutions of Wnt3a protein.

Primary cell harvest and culture. MEFs were isolated from E10.5 limb buds, incubated with 0.25% trypsin EDTA (Cellgro) at 37° C. for 5 minutes, triturated to dissociate clumps into single cells then cultured using a standard protocol. Briefly, the MEF cell suspension was diluted with media at a ratio of 1:9 v/v to neutralize trypsin then cells were plated in T25 flasks and incubated at 37° C., 5% CO₂ for two days with daily media changes. Thereafter cells were expanded in T150 flasks and cultured for up to p4. MEFs (50000 cells/cm²) were treated with Wnt3a at concentrations indicated) or Rspo2 at concentrations indicated, or Wnt3a+Rspo2 in DMEM supplemented with 10% FBS. After an incubation of 24 h, RNA was extracted and Axin2 expression was analyzed by qRT-PCR.

Histology and immunohistochemistry. Xgal staining detects the LacZ product, β-galactosidase. Tissues were fixed with 0.4% PFA (3 h, RT) then immersed in 30% sucrose at 4° C. Tissues were embedded in OCT and cryosectioned (10 μm) then fixed with 0.2% gluteraldehyde for 15 min and stained with Xgal at 37° C.

Histological staining. Samples were fixed 4.0% PFA, immersed in hematoxylin (30 s) then in Gomori trichome (10 m) or eosin (10 m) then differentiated in 0.2% acetic acid, dehydrated in ethanol and mounted.

TUNEL and BrdU. TUNEL (Roche, Indianapolis, Ind.) and BrdU stainings (Invitrogen, Carlsbad, Calif.) were performed as described by the manufacturers. Samples were developed using DAB (Vector laboratories). BrdU [1 mg/g of mouse weight] was administered via intraperitoneal injection 4 hours before sacrifice/tissue harvest (FIG. 2).

Imaging. Imaging was performed with Leica MZ16F fluorescent microscope; Adobe Photoshop CS5 and ImageJ software were used to calculate wound area. Ethidium bromide staining of whole tissues was used to capture fine morphological detail by briefly incubating tissues in a dilute solution of Ethidium bromide/PBS then photographed under UV light.

Measurement of hair length and thickness. Skin was harvested from injection cites using a 4 mm punch biopsy, without cutting the hairs. The biopsy was digested with 8 μg/ml collagenaseA/dispase in PBS at 37° C. for 3 hrs. Digested samples were vortexed with 10 ul hair conditioner and mounted on slides. Guard hairs were visualized using a 20× and 10× objectives and measured in photo shop CS5. Hair widths were calculated as hair area (using the magic wand tool) divided by hair length.

Quantitative RT-PCR. Tissue was harvested using a 3 mm punch biopsy, homogenized in TRIzol (Invitrogen), RNA quantified, and quantitative RT-PCR performed (Quantace Bioline, Taunton, Mass.). Expression levels were calculated using the 2″-(ddCt) method, normalized to GAPDH and converted to fold-expression compared to intact tissues from the Axin2^{LacZ/+} mice. The following primer sets were used: GAPDH, 5′-3′ (sense) acccagaagactgtggatgg and 5′-3′ (antisense) ggatgcagggatgatgttct. Axin2, 5′-3′ (sense) acacatgcagaaatgggtca and 5′-3′ (antisense) ggacgtctgtgacaagcaga. Left 5′-3′ (sense) aggagcccaaaagacctcat and 5′-3′ (antisense) cgtgcactcagctacgacat.

Statistical Analyses. In all quantitation experiments, results are expressed as the mean±SD. Statistical differences between sets of data were determined by using student t-tests in Microsoft Excel.

What is claimed is:

1. A cosmetic composition comprising:
an Rspo agonist in an amount effective to promote the anagen phase of hair production; and
a cosmetically acceptable vehicle.

2. The cosmetic composition of claim 1, wherein said promoting said anagen phase comprises increasing the length of said anagen phase.

3. The cosmetic composition of claim 1, wherein said promoting said anagen phase comprises increasing hair growth.

4. The cosmetic composition of claim 1, wherein said promoting anagen phase delays onset of catagen phase.

5. The cosmetic composition of any one of claims 1 to 4, wherein said Rspo agonist is a mammalian R-spondin polypeptide.

6. The cosmetic composition of claim 5, wherein said mammalian R-spondin polypeptide is a human R-spondin polypeptide.

7. The cosmetic composition of claim 5 or 6, wherein said R-spondin polypeptide is R-spondin2.

8. The cosmetic composition of any one of claims 1 to 4, wherein said Rspo agonist is at least about 30%, at least about 40%, at least about 50%, at least about 60%, at least about 70%, at least about 80%, at least about 90%, or at least about 95% sequence identical to at least one of the amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

9. The cosmetic composition of claim 8, wherein said Rspo agonist is as set forth in SEQ ID NO: 1 or SEQ ID NO: 2.

10. The cosmetic composition of claim 8, wherein said Rspo agonist comprises at least about 40%, at least about 50%, at least about 60%, at least about 70%, at least about 80%, at least about 90%, or at least about 95% sequence identity to at least one of the amino acid sequences as set forth in SEQ ID NO:1 or SEQ ID NO:2.

11. The cosmetic composition of any one of claims 1 to 4, wherein said Rspo agonist comprises at least a 10 amino acid sequence identical to SEQ ID NO: 1 or SEQ ID NO: 2.

12. The cosmetic composition of any one of claims 1 to 4, wherein said Rspo agonist comprises at least a 20 amino acid sequence identical to SEQ ID NO: 1 or SEQ ID NO: 2.

13. The cosmetic composition of any one of claims 1 to 4, wherein said Rspo agonist comprises no more than a 10 amino acid sequence identical to SEQ ID NO: 1 or SEQ ID NO: 2.

14. The cosmetic composition of any one of claims 1 to 4, wherein said Rspo agonist comprises no more than a 20 amino acid sequence identical to SEQ ID NO: 1 or SEQ ID NO: 2.

15. The cosmetic composition of any one of claims 1 to 4, wherein said Rspo agonist comprises from about 40% to about 95% sequence identity to at least one of the amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

16. The cosmetic composition of any one of claims 1 to 4, wherein said Rspo agonist comprises from about 40% to about 95% sequence identity to at least one of the amino acid sequences as set forth in SEQ ID NO:1 or SEQ ID NO:2.

17. The cosmetic composition of any one of claims 1 to 4, wherein said Rspo agonist comprises from about 40 to about 200 amino acids of a mammalian Rspo polypeptide.

18. The cosmetic composition of claim 17, wherein said Rspo agonist comprises from about 50 to about 180 amino acids of a mammalian Rspo polypeptide.

19. The cosmetic composition of any one of claims 1 to 18, wherein the composition further comprises one or more permeation enhancers.

20. The cosmetic composition of any one of claims 1 to 19, wherein the cosmetic composition comprises from about 0.01 to about 5.0 mg/mL of the Rspo agonist.

21. The cosmetic composition of claim 20, wherein the cosmetic composition comprises from about 0.5 to about 3 mg/mL of the Rspo agonist.

22. The cosmetic composition of any one of claims 1 to 21, wherein the cosmetically acceptable vehicle is selected from diluents, dispersants, and carriers or a combination thereof.

23. A patch for application to the skin, wherein the patch comprises said cosmetic composition of any one of claims 1 to 22.

24. A method for treating a condition of the skin, scalp or hair of a subject in need thereof, comprising applying to said skin, scalp or hair of said subject said cosmetic composition of any one of claims 1 to 22.

25. The method of claim 24, wherein said condition is alopecia.

26. The method of claim 24, wherein said condition is androgenic alopecia.

27. The method of claim 24, wherein said condition is caused by a vitamin deficiency, an iron deficiency, infection, chemotherapy, anabolic steroids, oral contraceptives or trauma.

28. The method of claim 24, wherein said subject is human.

29. The method of claim 24, wherein said applying comprises one or more techniques selected from non-cavitational ultrasound, electroporation, cavitational ultrasound, thermal ablation, and microdermabrasion to enhance transdermal delivery of said Rspo agonist.

30. The method of claim 24, wherein said applying comprises injecting said Rspo agonist subcutaneously.

31. The method of claim 30, wherein said injecting is performed with one or more microneedles.

32. The method of claim 31, wherein said injecting is performed with an array of microneedles.

33. The method of any of claims 24 to 32, wherein said cosmetic composition is applied to said scalp of said subject.

34. The method of claim 24, wherein said applying comprises contacting said scalp of said subject with said patch of claim 23.

35. The method of claim 24, wherein said applying comprises applying the composition at least once a month.

36. The method of claim 35, wherein applying comprises applying the composition at least once a week.

37. The method of claim 35, wherein applying comprises applying the composition at least once a day.


SEQUENCE LISTING (9 sequences)

SEQ ID NO: 1 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 2 - protein, 179 aa, Homo sapiens
MQFRLFSFALIILNCMDYSHCQGNRWRRSKRGCRIENCDSCFSKDFCTKCKVGFYLHRGRCFDECPDGFAPLEETMECVGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 3 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 4 - protein, 272 aa, Homo sapiens; identical to SEQ ID NO: 3

SEQ ID NO: 5 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 6 - protein, 236 aa, Homo sapiens
MIFRVSAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 7 - protein, 200 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGQKRRKGGQGRRENANRNLARKESKEAGAGSRRRKGQQQQQQQGTVGPLTSAGPA

SEQ ID NO: 8 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 9 - protein, 172 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQERSPGQKKGRKDRRPRKDRKLDRRLDVRPRQPGLQP